(19) United States
(12) Patent Application Publication
(10) Pub. No.: US 2014/0314746 A1
(43) Pub. Date: Oct. 23, 2014

(54) METHODS FOR TREATING OR PREVENTING FIBROSIS IN SUBJECTS AFFLICTED WITH SCLERODERMA

(71) Applicant: Philadelphia Health and Education Corporation, d/b/a Drexel University College of Medicine, Philadelphia, PA (US)

(72) Inventors: CAROL M. ARTLETT, WAYNE, PA (US); PETER D. KATSIKIS, MERION STATION, PA (US)

(73) Assignee: Philadelphia Health and Education Corporation, d/b/a Drexel University College of Medicine, Philadelphia, PA (US)

(21) Appl. No.: 14/204,235

(22) Filed: Mar. 11, 2014

Related U.S. Application Data
(60) Provisional application No. 61/779,883, filed on Mar. 13, 2013.

(51) Int. Cl. A61K 31/64; A61K 45/06; A61K 39/395; A61K 31/365; A61K 38/16

(52) U.S. Cl. CPC A61K 31/64; A61K 31/365; A61K 38/16; A61K 39/3955; A61K 45/06
USPC 424/133.1; 514/593; 514/468; 514/1.1; 424/158.1; 424/145.1; 514/44 A; 424/146.1; 514/266.22; 514/110; 514/252.19; 564/41; 549/458; 530/350; 530/389.1; 536/24.5

(57) ABSTRACT

The invention includes novel methods of treating or preventing fibrosis in a subject afflicted with scleroderma, comprising administering to the subject a therapeutically effective amount of an agent that inhibits formation of at least one inflammasome signaling product in the subject.

### CROSS REFERENCE TO RELATED APPLICATION

The present application is entitled to priority under 35 U.S.C. §119(e) to U.S. Provisional Patent Application 61/779,883, filed Mar. 13, 2013, which is hereby incorporated by reference in its entirety herein.

### BACKGROUND OF THE INVENTION

Systemic sclerosis (SSc) or scleroderma is a chronic, progressive connective tissue disease of unknown etiology characterized by uncontrolled fibrosis, which is directly related to the morbidity and mortality of the disease (Varga, 2008, Bull NYU Hosp Jt Dis 66:198-202). Affecting less than 500,000 patients in the U.S., mostly women, this disease is characterized by excessive deposition of collagen in the skin and visceral organs. There is no effective treatment for SSc, and the outcome for patients afflicted with this disease is death.

SSc is characterized by tissue injury (of unknown cause) that leads to the excessive deposition of collagen in what can be considered to be an abnormal balance between matrix production and degradation. This abnormal balance results in the excessive accumulation of collagen and a progressive fibrotic pathology that modulates the morbidity and mortality of SSc. In fibrotic SSc lesions, persistently activated fibroblasts called myofibroblasts mediate the excessive secretion of collagen in both the dermis and visceral organs (Atamas et al., 2003, Am J Respir Cell Mol Biol 29:743-9; Kirk et al., 1995, J Biol Chem 270:3423-8; Varga & Jimenez, 1995, Ann Intern Med 122:60-2). Myofibroblasts are phenotypically characterized by increased α-smooth muscle actin (α-SMA) (Hinz et al., 2003, Mol Biol Cell 14:2508-19) and these cells are involved in the extensive tissue damage and tissue remodeling (Abraham et al., 2007, Curr Rheumatol Rep 9:136-43). The cytokine TGF-β1 has well established roles in tissue repair and collagen production and plays a direct role on fibroblast differentiation to myofibroblasts (Tomasek et al., 2002, Nature Rev 3:349-63; Gu et al., 2007, Acta Pharmacol Sin 28:382-91) and inducing the increased secretion of collagen by these specialized cells (Pohlers et al., 2009, Biochim Biophys Acta 1792:746-56).

The inflammasomes comprise of a family of cytosolic pattern recognition receptors called NOD-like receptors (NLR) that are involved in innate immune recognition of pathogen associated molecular patterns (PAMPs) as well as intracellular and extracellular damage associated molecular patterns (DAMPs). NLRP3 is the most extensively studied receptor and is activated by various stimuli including microbial derived products (Dostert et al., 2009, PLoS One 4:e6510; Gurcel et al., 2006, Cell 126:1135-45; Chu et al., 2009, J Leukoc Biol 86:1227-38; Thomas et al., 2009, Immunity 30:566-75), environmental factors (Dostert et al., 2009, PLoS One 4:e6510; Hornung et al., 2008, Nat Immunol 9:847-56; Cassel et al., 2008, Proc Natl Acad Sci, USA 105:9035-40) and endogenous molecules (Yamasaki et al., 2009, J Biol Chem 284:12762-71; Salminen et al., 2008, J Cell Mol Med 12:2255-62; Mariathasan et al., 2006, Nature 440:228-32; Gasse et al., 2009, Am J Respir Crit Care Med 179:903-13). Once activated, the NLR aggregates with the adaptor molecule apoptosis speck-like protein containing a CARD (ASC) to serve as a scaffold for the assembly of the inflammasome (Martinon et al., 2007, Semin Immunopathol 29:213-29). The assembly of the inflammasome activates caspase-1, which then participates in substrate cleavage of pro-IL-1β and pro-IL-18, into their active forms, inducing their secretion from the cell.

There is a need in the art to identify a novel method of preventing or controlling disregulated collagen expression in a subject afflicted with scleroderma. Such method would inhibit the initiating event that leads to increased collagen secretion in the subject. The present invention fulfills this need.

### BRIEF SUMMARY OF THE INVENTION

The invention includes a method of treating or preventing fibrosis in a subject afflicted with scleroderma. The method comprises administering to the subject a therapeutically effective amount of a pharmaceutical composition comprising an agent that inhibits formation of at least one inflammasome signaling product in the subject, whereby the fibrosis in the subject is treated or prevented.

In one embodiment of the invention, the agent that inhibits formation of at least one inflammasome signaling product is selected from the group consisting of glyburide, parthenolide, BAY 11-7082, colchicine, a caspase-1 inhibitor, an IL-1β-depleting agent, and any combinations thereof.

In another embodiment, the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, a ribozyme, an antisense, an aptamer, a peptidomimetic, a small molecule, and any combination thereof.

In yet a further embodiment, the IL-1β antibody comprises an antibody selected from the group consisting of a polyclonal antibody, monoclonal antibody, humanized antibody, synthetic antibody, heavy chain antibody, human antibody, biologically active fragment of an antibody, and any combinations thereof.

In an additional embodiment, the caspase-1 inhibitor is selected from the group consisting of a caspase-1 antibody, siRNA, ribozyme, antisense, aptamer, peptidomimetic, small molecule, and a combination thereof, where when a caspase-1 antibody, the antibody is selected from the group consisting of a polyclonal antibody, monoclonal antibody, humanized antibody, synthetic antibody, heavy chain antibody, human antibody, biologically active fragment of an antibody, and any combinations thereof.

In another aspect of the invention, the method comprises further administering to the subject an additional anti-scleroderma composition. The anti-scleroderma composition comprises a vasodilator, an endothelin receptor antagonist, an angiotensin converting enzyme inhibitor, an immunosuppressive agent, halofuginone, CAT-192, cyclophosphamide and rabbit antithymocyte globulins, dasatinib, fludarabine, imatinib mesylate (Gleevec), nilotnib (Tasigna) or rituximab.

In one embodiment, the agent and the anti-scleroderma composition are co-administered to the subject. In another embodiment, the agent and the anti-scleroderma composition are co-formulated and co-administered to the subject.

In a further embodiment, the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combination thereof.

In one embodiment, the subject is a mammal, and in another embodiment, the mammal is a human.

Also included in the invention is a kit comprising an agent that inhibits formation of at least one inflammasome signaling product in a subject, an applicator, and an instructional material for use thereof, wherein the agent that inhibits formation of at least one inflammasome signaling product is selected from the group consisting of glyburide, parthenolide, BAY 11-7082, colchicine, a caspase-1 inhibitor, an IL-1β-depleting agent, and any combinations thereof; wherein the instructional material comprises instructions for preventing or treating fibrosis in a subject afflicted with scleroderma, wherein the instructional material recites that the agent is to be administered to the subject in an amount sufficient to inhibit formation of at least one inflammasome signaling product in the subject, whereby the fibrosis in the subject is treated or prevented.

In one embodiment, the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, ribozyme, an antisense, an aptamer, a peptidomimetic, a small molecule, and any combination thereof. In another embodiment, the caspase-1 inhibitor is selected from the group consisting of a caspase-1 antibody, siRNA, ribozyme, antisense, aptamer, peptidomimetic, small molecule, and a combination thereof. In further embodiments, the subject is a mammal or a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1D, illustrates the non-limiting inflammasome gene expression signature in SSc. The gene expression signatures of three SSc dermal fibroblast cell lines (SSc1, SSc3, SSc4) were compared to the gene expression signatures of three normal dermal fibroblast cell lines (C1, C2, C3) were analyzed by RT-PCR for 84 genes involved in the inflammasome or downstream inflammasome mediated signaling. The expression of each gene has been centered on its median expression value across all samples analyzed. Measurements that were above the median was colored red and those below the median was colored green and the intensity of the color was directly proportional to the fold change. FIG. 1A: Heat map of expressed genes. FIG. 1B: Relative caspase-1 expression in SSc (n=3) vs. control (n=3). FIG. 1C: Western blot for AIM2 and CARD6 proteins normalized to relative expression of β-actin. FIG. 1D: Western blot of NLRP3 protein normalized to relative expression of β-actin. Black bars=normal fibroblasts, grey bars=SSc fibroblasts.

FIG. 2, comprising FIGS. 2A-2G, illustrates the inhibition of IL-1β and IL-18 secretion by SSc dermal and SSc lung fibroblasts. SSc dermal fibroblasts (SSc1, SSc2, SSc3, SSc4), control dermal fibroblasts (C1, C2, C3, C4), SSc lung fibroblasts (SSc12, SSc14, SSc15), and normal lung fibroblasts (NL32, NL34, NL35) were cultured +ZYVAD(OMe)-FMK for 48 h and media was collected and assayed for secreted IL-1β and IL-18. FIG. 2A: IL-1β secretion by SSc dermal fibroblasts and normal dermal fibroblasts; FIG. 2B: IL-1β secretion by SSc lung fibroblasts and normal lung fibroblasts; FIG. 2C: IL-18 secretion by SSc dermal fibroblasts and normal dermal fibroblasts; FIG. 2D: IL-1β secretion by SSc lung fibroblasts and normal lung fibroblasts; FIG. 2E: Western blot for IL-18 and IL-1β in the media that was assayed by ELISA; FIG. 2F: ELISA for IL-1β in SSc cells treated with siRNA for caspase-1; FIG. 2G: ELISA for IL-18 in SSc cells treated with siRNA for caspase-1. Black bars=normal fibroblasts; grey bars=SSc fibroblasts; white bars=fibroblasts with Z-YVAD(OMe)-FMK; grey textured bars=SSc fibroblasts+siRNA for caspase-1. Data were presented as averages of each sample tested in at least duplicate±SEM.

FIG. 3, comprising FIGS. 3A-3I, illustrates the finding that collagen expression in SSc dermal and lung fibroblasts is controlled by caspase-1. SSc dermal fibroblasts (SSc1, SSc2, SSc3, SSc4) and SSc lung fibroblasts (SSc12, SSc14, SSc15) were treated with Z-YVAD(OMe)-FMK (A,B,D-I) or caspase-1 siRNA (C) in culture for 48 h and media collected and assayed for hydroxyproline, and COL1A1 and COL3A1 secretion. FIG. 3A: Secretion of hydroxyproline by SSc dermal fibroblasts; FIG. 3B: Secretion of hydroxyproline by SSc lung fibroblasts; FIG. 3C: Secretion of hydroxyproline by SSc dermal fibroblasts treated with caspase-1 siRNA; FIG. 3D: Western blots for COL1A1 and COL3A1 protein secreted by SSc dermal fibroblasts; FIG. 3E: ImageJ densitometry for COL1A1 of the Western blot in D; FIG. 3F: ImageJ densitometry for COL3A1 of the Western blot in D; FIG. 3G: Western blots for COL1A1 and COL3A1 protein secreted by SSc lung fibroblasts; FIG. 3H: ImageJ densitometry for COL1A1 of the Western blot in G; FIG. 3I: ImageJ densitometry for COL3A1 of the Western blot in G; Black bars=normal fibroblasts; grey solid bars=SSc fibroblasts; white bars=fibroblasts with Z-YVAD(OMe)-FMF; grey textured bars=SSc dermal fibroblasts+caspase-1 siRNA; AU=arbitrary units. Data were presented as the average for each sample tested in at least duplicate±SEM.

FIG. 4, comprising FIGS. 4A-4D, illustrates the finding that α-smooth muscle actin expression in SSc myofibroblasts is decreased by caspase-1 inhibition. SSc dermal fibroblasts (SSc1, SSc2, SSc3, SSc4) were cultured for 48 h in chamber slides with Z-YVAD-(OMe)-FMK and stained for α-smooth muscle actin (α-SMA) and f-actin. All images (red for f-actin, green for α-SMA; and triple for simultaneous detection of f-actin, α-SMA and nuclei that are stained blue with DaPI) were captured at 6 sec, gain=2, and gamma=0.5. FIG. 4 A: Immunofluorescence of SSc dermal myofibroblasts treated with or without Z-YVAD-(OMe)-FMK. FIG. 4B: Quantification of mean fluorescent intensity of α-SMA in myofibroblasts treated with or without Z-YVAD-(OMe)-FMK normalized to f-actin expression. Data is presented as the average of MFI in at least 5 myofibroblasts in each sample±SEM. FIG. 4C: Western blot of α-SMA. FIG. 4D is a graph. Grey bars=SSc fibroblasts; white bars=SSc fibroblasts with Z-YVAD(OMe)-FMF; grey textured bars=SSc dermal fibroblasts+caspase-1 siRNA. Scale bar=100 μm.

FIG. 5, comprising FIGS. 5A-5C, illustrates the finding that in vivo administration of bleomycin requires the inflammasome to mediate fibrosis. C57Bl/6 control mice (n=7), NLRP3−/− (n=8), and ASC−/− (n=4) mice were treated with 100 μg bleomycin daily for 28 days. FIG. 5A: Masson's trichrome stain of representative skin and lung tissues from C57Bl/6, NLRP3−/−, and ASC−/− mice after bleomycin treatment and PBS treatments, and from untreated mice. FIG. 5B: Dermal thickness between the PBS (white bars) and bleomycin (black bars) for each genetic background. Gray bars correspond to the skin thickness of untreated C57BL/6 mice. Scale bar=100 μm. FIG. 5C: Hydroxyproline secreted from mouse dermal fibroblasts derived from C57BL/6, NLRP3−/−, and ASC−/− mice treated with bleomycin (BLM) and/or BLM and (YVAD). Three independent experiments in triplicate were performed and data is presented as an average±SEM.

FIG. 6 is a schematic non-limiting representation of the experiments described herein.

FIG. 7 is a series of graphs illustrating the finding that inhibition of CASP1 abrogates IL-1β, IL-18, and hydroxyproline secretion by SSc fibroblasts. SSc dermal fibroblasts (n=4), control dermal fibroblasts (n=3), SSc lung fibroblasts (n=3), and normal lung fibroblasts (n=3) were cultured for 48 h and media was collected and assayed for secreted IL-1β, IL-18 and hydroxyproline. SSc fibroblasts were treated with 20 μM YVAD for 48 h and also assayed for IL-1β and IL-18. Normal fibroblasts (black bars), untreated SSc fibroblasts (gray bars), YVAD treated fibroblasts (green bars). Data presented as averages of each sample tested in at least duplicate±SEM. ^{φ}p=0.012, ^{Ω}p=0.001, ^{Ψ}p=0.003, ^{Σ}p=0.001, ^{≅}p=0.011.

FIG. 8, comprising FIGS. 8A-8C, illustrates the finding that α-smooth muscle actin (α-SMA) expression in SSc myofibroblasts is decreased with CASP1 inhibition. SSc dermal fibroblasts (n=4) were cultured for 48 h with YVAD and stained for α-SMA and f-actin. All images (red for f-actin, green for α-SMA, and triple for simultaneous detection of f-actin and α-SMA) were captured at 6 sec, gain=2, gamma=0.5. FIG. 8A: Fluorescence of SSc dermal myofibroblasts. FIG. 8B: Quantification of mean fluorescent intensity (MFI) of α-SMA staining in myofibroblasts by ImageJ, presented as an average of at least 6 myofibroblasts in each sample±SEM. FIG. 8C: Western blot of α-SMA. Grey bars=SSc fibroblasts; White bars=SSc fibroblasts with YVAD. Scale bar=100 μm. *p<0.001, **p=0.008.

FIG. 9 is a set of graph illustrating the finding that glyburide and parthenolide significantly reduce hydroxyproline secretion by SSc fibroblasts. SSc fibroblasts were cultured in 5 ml of media with 100 μM glyburide (G) or 10 μM parthenolide (P). After 24 h the media was collected and the fibroblasts given an additional 5 ml of media with 100 μM glyburide or 10 μM parthenolide and cultured for a further 24 h. Media from both time points was assayed for hydroxyproline. Three independent experiments were performed in and assayed in triplicate. Data is presented as means±SEM. G=glyburide, P=parthenolide.

FIG. 10 is a graph illustrating the finding that bleomycin (BLM) induced collagen expression is abrogated with YVAD. Mouse dermal fibroblasts derived from C57BL/6 (B6), NLRP3−/−, and ASC−/− mice were treated with 1 μM BLM (B) for 48 h, and B6 fibroblasts were also treated with 1 μM BLM and 20 μM YVAD (Y). Hydroxyproline was measured. The data is presented as an average±SEM of three independent experiments performed in triplicate. *p=0.036, **p=0.008.

### DETAILED DESCRIPTION OF THE INVENTION

The present invention relates to the treatment or prevention of fibrosis in scleroderma (SSc). As demonstrated herein, an inflammasome is involved in SSc pathology. AIM2, NLRP3, and NOD2 inflammasomes are upregulated in SSc and inhibition of inflammasome activated CASP1 with Z-YVAD-FMK abrogated secretion of collagen (measured as hydroxyproline), IL-1β, and IL-18.

Based on the unexpected discovery described herein that the inhibition of caspase-1 abrogated collagen expression in SSc fibroblasts, compositions useful in the treatment of SSc were identified. These compositions inhibit at least one inflammasome signaling product. In one embodiment, the at least one inflammasome signaling product comprises IL-1β.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element.

A “subject” or “individual” or “patient,” as used therein, can be a human or non-human mammal. Non-human mammals include, for example, livestock and pets, such as ovine, bovine, porcine, canine, feline and murine mammals. Preferably, the subject is human.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “glyburide” or “glibenclamide” refers to 5-chloro-N-(4-[N-(cyclohexylcarbamoyl)sulfamoyl]phenethyl)-2-methoxybenzamide or a salt or solvate thereof.

As used herein, the term “parthenolide” refers to (1aR,7aS,10aS,10bS)-1a,5-dimethyl-8-methylene-2,3,6,7,7a,8,10a,10b-octahydrooxireno[9,10]cyclodeca[1,2-b]furan-9(1aH)-one or a salt or solvate thereof.

As used herein, the term “BAY 11-7082” refers to (E)-3-[(4-methyl-phenyl)sulfonyl]-2-propenenitrile or a salt or solvate thereof.

As used herein, the term “colchicine” refers to N-[(7S)-1,2,3,10-tetramethoxy-9-oxo-5,6,7,9-tetrahydrobenzo[a]heptalen-7-yl]acetamide or a salt or solvate thereof.

As used herein, the term “SSc” refers to systemic sclerosis or scleroderma.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “treatment” or “treating” is defined as the application or administration of a therapeutic agent, i.e., a compound useful within the invention (alone or in combination with another pharmaceutical agent), to a subject, or application or administration of a therapeutic agent to an isolated tissue or cell line from a subject (e.g., for diagnosis or ex vivo applications), who has the disease, a symptom of the disease or the potential to develop the disease, with the purpose to cure, heal, alleviate, relieve, alter, remedy, ameliorate, improve or affect the disease, the symptoms of the disease or the potential to develop the disease. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the terms “effective amount” or “therapeutically effective amount” or “pharmaceutically effective amount” of a compound are used interchangeably to refer to the amount of the compound that is sufficient to provide a beneficial effect to the subject to which the compound is administered. The term to “treat,” as used herein, means reducing the frequency with which symptoms are experienced by a patient or subject or administering an agent or compound to reduce the severity with which symptoms are experienced. An appropriate therapeutic amount in any individual case may be determined by one of ordinary skill in the art using routine experimentation. By the term “specifically bind” or “specifically binds,” as used herein, is meant that a first molecule (e.g., an antibody) preferentially binds to a second molecule (e.g., a particular antigenic epitope), but does not necessarily bind only to that second molecule.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The term “antibody,” as used herein, refers to an immunoglobulin molecule able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody generated using recombinant DNA technology, such as, for example, an antibody expressed by a bacteriophage as described herein. The term should also be construed to mean an antibody generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopts highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

As used herein “endogenous” refers to any material from or produced inside an organism, cell, tissue or system.

As used herein, the term “exogenous” refers to any material introduced from or produced outside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “protein” typically refers to large polypeptides. The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus. As used herein, a “peptidomimetic” is a compound containing non-peptidic structural elements that is capable of mimicking the biological action of a parent peptide. A peptidomimetic may or may not comprise peptide bonds.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

In one embodiment, a composition useful within the methods of the invention comprises at least one caspase-1 inhibitor. Such agent reduces the expression, production and/or circulating concentration of caspase-1 in a subject. In one embodiment, the agent is an antibody that binds to and neutralizes caspase-1. In another embodiment, the agent is a chemical compound that inhibits formation of caspase-1. In yet another embodiment, the agent reduces the expression or production of caspase-1 in the subject. Agents that reduce the expression, production and/or circulating concentration of IL-1β by any physiological mechanism are considered useful within the methods of the invention. Non-limiting examples of caspase-1 inhibitors contemplated within the methods of the invention are any caspase-1 antibody, siRNA, ribozyme, antisense, aptamer, peptidomimetic, small molecule, and a combination thereof.

In one embodiment, a composition useful within the methods of the invention comprises at least one IL-1β-depleting agent. Such agent reduces the expression, production and/or circulating concentration of IL-1β in a subject. In one embodiment, the agent is an antibody that binds to and neutralizes IL-1β. In another embodiment, the agent is a chemical compound that inhibits formation of IL-1β. In yet another embodiment, the agent reduces the expression or production of IL-1β in the subject. In yet another embodiment, the agent is selected from the group consisting of anakinra, XOMA-052, AMG-108, canakinumab, rilonacept, K-832, CYT-013-IL1bQb, LY-2189102, dexamethasone, interferon-gamma, pentoxifylline, and any combinations thereof. Agents that reduce the expression, production and/or circulating concentration of IL-1β by any physiological mechanism are considered useful within the methods of the invention.

Non-limiting examples of IL-1β-depleting agents contemplated within the methods of the invention are:

Dexamethasone (8S,9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one) or a salt thereof: a known inhibitor of IL-1β production (Kern et al., 1998, J. Clin. Invest. 81:237-244);

Canakinumab (also known as Ilaris®, Novartis; previously ACZ885; Dhimolea, 2010, Mabs 2(1):3-13): http://en.wikipedia.org/wiki/Canakinumab-cite_note-0a human monoclonal antibody targeted at interleukin-1 beta. It has no cross-reactivity with other members of the interleukin-1 family, including interleukin-1 alpha (Lachmann et al., 2009, New Engl. J. Med. 360(23):2416-25). Canakinumab was approved for the treatment of cryopyrin-associated periodic syndromes (CAPS) by the FDA on June 2009 and by the European Medicines Agency in October 2009. Canakinumab is also in clinical trials as a possible treatment for chronic obstructive pulmonary disease (Yasothan & Kar, 2008, Nat. Rev. Drug Discov. 7(4):285).

Rilonacept (also known as IL-1 Trap or Arcalyst®, Regeneron): an interleukin 1 inhibitor (Drug News Perspect. 21(4): 232). Rilonacept is a dimeric fusion protein consisting of the extracellular domain of human interleukin-1 receptor and the FC domain of human IgG1 that binds and neutralizes IL-1. Rilonacept is used for the treatment of cryopyrin-associated periodic syndromes (CAPS).

AMG-108: a fully human monoclonal antibody that targets inhibition of the action of interleukin-1 (Cardiel et al., Arthritis Res. Ther. 12(5):R192).

Anakinra (also known as Kineret®, Amgen): Anakinra is an IL-1 receptor antagonist (So et al., 2007, Arthritis Res. Ther. 9(2):R28). Anakinra is a recombinant, non-glycosylated version of human IL-1 receptor antagonist prepared from cultures of genetically modified E. coli. Anakinra blocks the biologic activity of naturally occurring IL-1, by competitively inhibiting the binding of IL-1 to the Interleukin-1 type receptor.

Interferon-gamma: known to selectively inhibit IL-1β gene expression (Chujor et al., 1996, Eur. J. Immun. 26:1253-1259).

Pentoxifylline: a known inhibitor of the synthesis of IL1-β (Roy et al., 2007, J. Toxicol. Environ. Health B Crit. Rev. 10(4):235-57; Zargari, 2008, Dermat. Online J. 14(11):2).

XOMA-052 (also known as gevokizumab): a potent anti-IL-1β humanized neutralizing antibody (Owyang et al., 2011, mAbs 3(1):49-60; U.S. Pat. No. 7,531,116 to Masat et al.). XOMA-052 has a 300 femtomolar binding affinity for human IL-1β and an in vitro potency in the low picomolar range. XOMA-052 has been shown to be active in mouse models of acute gout.

K-832 (also known as 2-benzyl-5-(4-chlorophenyl)-6-[4-(methylthio)phenyl]-2H-pyridazin-3-one): this compound has high inhibitory activity against production of interleukin-1β (i.e., acts as a IL-1β secretion inhibitor), and is being tested as a preventive and therapeutic drug for immune, inflammatory, and ischemic diseases (U.S. Pat. No. 6,348,468 to Ohkuchi et al.; Tabunoki et al., 2003, Arthritis Rheum. 48 (Suppl. S555); Miura et al., 2010, Eur. J. Pharm. Biopharm. 76(2):215-221).

CYT-013-IL1bQb (Cytos Biotechnology AG): a IL-1β vaccine (ClinicalTrials.gov., NCT00924105; http://www dot clinicaltrials dot gov/ct2/show/NCT00924105).

LY-2189102 (Eli Lilly): a humanized IgG4 monoclonal anti-IL1β antibody in development for the treatment of diabetes, with a binding affinity of 2.8 pM, and a half-life and bioavailability of 20.3 days and 55%, respectively, after SC administration to healthy volunteers. LY2189102 was recently studied in a Phase II study in T2DM patients (CT registry NCT00942188; ClinicalTrials.gov., 2011, “A safety and pharmacokinetics study in patients with rheumatoid arthritis”, at: http://www dot clinicaltrials dot gov/ct2/show/NCT00380744; ClinicalTrials.gov., 2011, “A study for patients with rheumatoid arthritis on methotrexate (MTX) with an inadequate response to TNF inhibitor therapy”, at: http://www dot clinicaltrials dot gov/ct2/show/NCT00689728.

Further non-limiting examples of IL-1β-depleting agents contemplated within the methods of the invention are any IL-1β antibody, siRNA, ribozyme, antisense, aptamer, peptidomimetic, small molecule, and a combination thereof.

### Description

Systemic sclerosis (SSc) is a serious disease of unknown cause characterized by excessive deposition of collagen in the skin and visceral organs. There is no effective treatment for SSc, and the outcome for afflicted subjects is death.

As demonstrated herein, there is an increased expression of AIM2, NLRP3, and NOD2 inflammasomes in SSc cells and also an increase in CASP1-dependent collagen secretion.

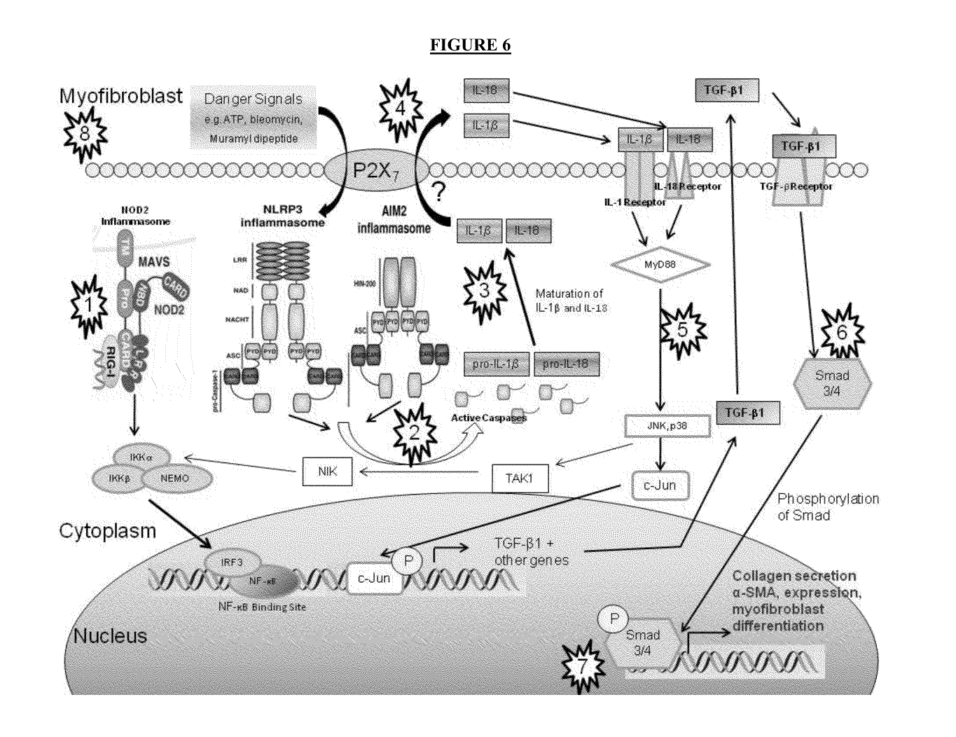

The studies described herein shed light on the role of the inflammasome in cell populations found in fibrotic lesions of subjects with SSc. These studies allow one to understand the contribution of the inflammasome in SSc and the potential role that these signaling proteins play in fibrosis. The studies also allow one to determine how inflammasome-mediated downstream signaling molecules, IL-1β and IL-18, affect TGF-β1 expression; and determine whether autocrine signaling by IL-1β, IL-18, and/or TGF-β1 maintains chronic expression of the inflammasome. The activation of purinergic receptors and pannexin-1, known to lead to IL-1β and IL-18 secretion, is investigated. Moreover, the ability of therapeutic compositions that modulate the inflammasome signaling to down-regulate collagen in a mouse model of fibrosis that mimics SSc is investigated. A non-limiting illustration of the experiments is presented in FIG. 6.

In a non-limiting aspect, inflammasome signaling is a crucial component of SSc fibrosis, and leads to the chronic activation of CASP1 and the subsequent secretion of IL-1β and IL-18. The ensuing cytokine release modulated by inflammasome signaling leads to the expression of the profibrotic cytokine TGF-β1, the conversion of fibroblasts to myofibroblasts, and the enhanced deposition of collagen.

Since fibrosis is the hallmark of SSc, understanding the involvement of the inflammasome that drives the profibrotic events in SSc is important to understanding this pathological feature. The overview of the experiments reported herein is summarized in FIG. 6. The experiments described herein address the following issues: whether both skin and lung SSc fibroblasts have autonomous inflammasome activation; whether the other inflammatory caspases are activated; whether inhibition of CASP4 and CASP5 abrogate IL-1β and IL-18 secretion; whether fibrosis can be initiated through the P2X₇ purinergic receptor; whether IL-1β and IL-18 autocrine signal induce TGF-β1; whether TGF-β1 activates the canonical Smad pathway; whether a functional inflammasome is required for TGF-β1 signaling; whether TGF-β1 is required for fibrosis in this model, or whether IL-1β or IL-18 can induce collagen and myofibroblast differentiation; whether inhibition of the inflammasome and/or downstream inflammasome mediated signaling products can prevent fibrosis in a mouse model of SSc. In one embodiment, the fact that estrogen-responsive binding box B protein is not only secreted by the inflammasome but also enhances the secretion of IL-1β causes women to be pre-disposed to autoimmune diseases.

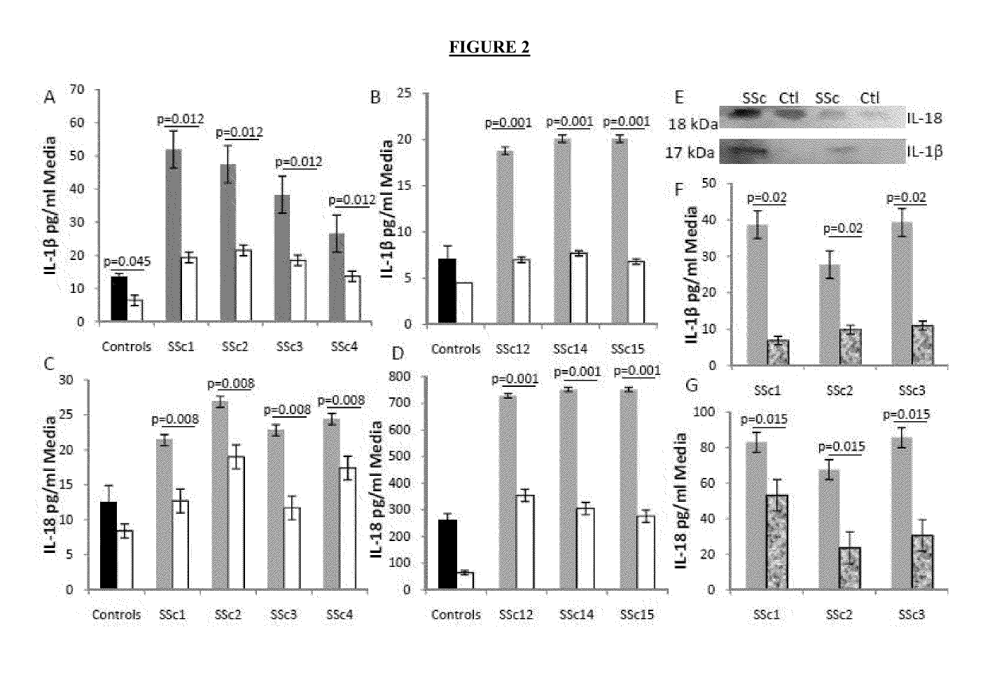

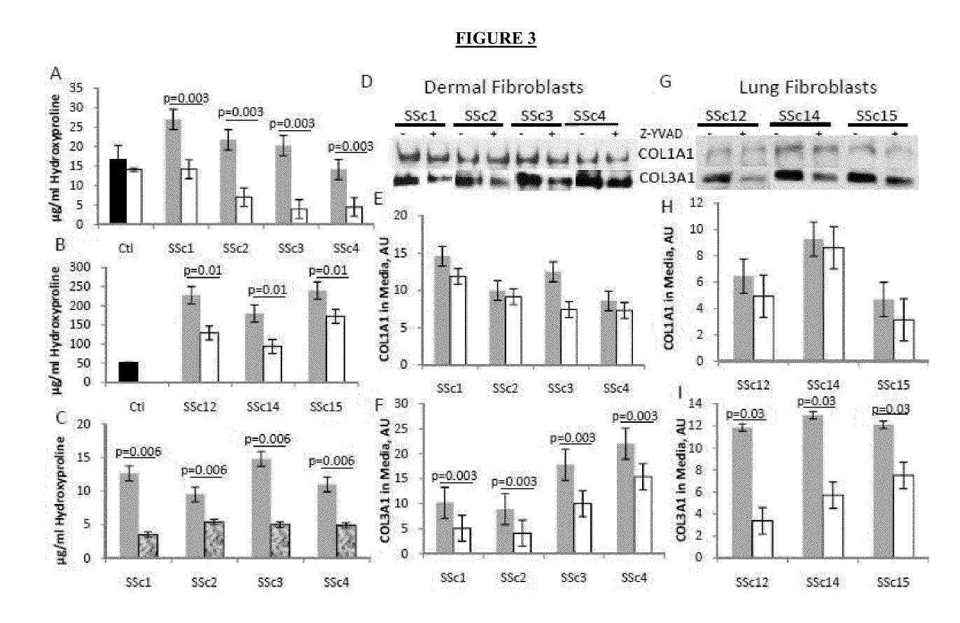

To date, nothing is known about the role of the inflammasome in SSc. In one aspect, innate immune signaling mediated by functional CASP1 leads to the secretion of IL-1β and IL-18, and this controls the increased deposition of collagen (FIG. 2) and myofibroblast differentiation (FIG. 3). This data strongly connects the activation of the inflammasome with SSc fibrosis. Once IL-1β and IL-18 are secreted from a cell, they are able to autocrine signal by engaging their specific receptors (IL-1R and IL-18R). IL-18 is a potent inducer of IFNγ but can also induce IL-4 expression, and facilitate TH2 differentiation of T cells. IL-6 is elevated in SSc serum, is over expressed in affected fibroblasts isolated from SSc patients, and has increased expression on the inflammasome arrays. IL-1β induces the expression of IL-6, and this further supports to role of the inflammasome signaling complex in the pathogenesis of SSc.

The present studies address the following questions regarding SSc:

Inflammasome Participation in Disease:

AIM2, NLRP3 and NOD2 have increased expression in SSc dermal fibroblasts, and lung fibrosis is an important cause of mortality in SSc patients. In one embodiment, SSc lung fibroblasts (and normal fibroblasts stimulated with bleomycin) may demonstrate that same pattern of inflammasome expression as SSc dermal fibroblasts. In another embodiment, this elevated expression is specific for myofibroblasts. In another embodiment, fibroblasts also express high levels of AIM2, NLRP3, and NOD2. In yet another embodiment, AIM2, NLRP3, and NOD2 inflammasomes are activated. Inflammasome activation can also activate other inflammatory caspases, and it is important to know if CASP4 and CASP5 are also active and if they contribute to the secretion of IL-1β and IL-18, and eventually collagen, from SSc fibroblasts. Because myofibroblasts have many similarities to macrophages (e.g. phagocytosis and antigen presentation), and macrophages require two stimuli to induce inflammasome activation, myofibroblasts may also require two stimuli.

Link Between Inflammasome Activation and TGF-β1:

The finding that three inflammasome subtypes were all up-regulated in the SSc fibroblasts was surprising. Any functional integration of inflammasome signaling among the three major forms of inflammasomes is unknown in fibroblasts vs. myofibroblasts, or SSc vs. normal cells. The various contributions of the three inflammasome subsets are investigated. Autocrine signaling by IL-1β or IL-18 that promotes the expression of TGF-β1 is likely but unproven in two ways: it is not known if a cell from lesional SSc skin responds to its own IL-1β or IL-18 upregulating NF-kB expression, in an autocrine fashion, and it is not known if TGF-β1 is necessary for the consequent fibrosis as a result of this IL-1β pathway.

Initiating Event is SSc is Unknown:

In one embodiment, P2X7 promotes inflammasome activation in SSc fibroblasts, and that inhibition of this receptor abrogate collagen secretion. While it is known that P2X7 activation can induce the opening of the pannexin-1 hemichannel, this important feature has not yet been studied in fibroblasts. It is also unknown if an inherent difference exists between SSc and unaffected dermal cells in culture to respond more aggressively to P2X7 stimulation.

Potential of Existing Compositions to Block or Even Alleviate Fibrosis:

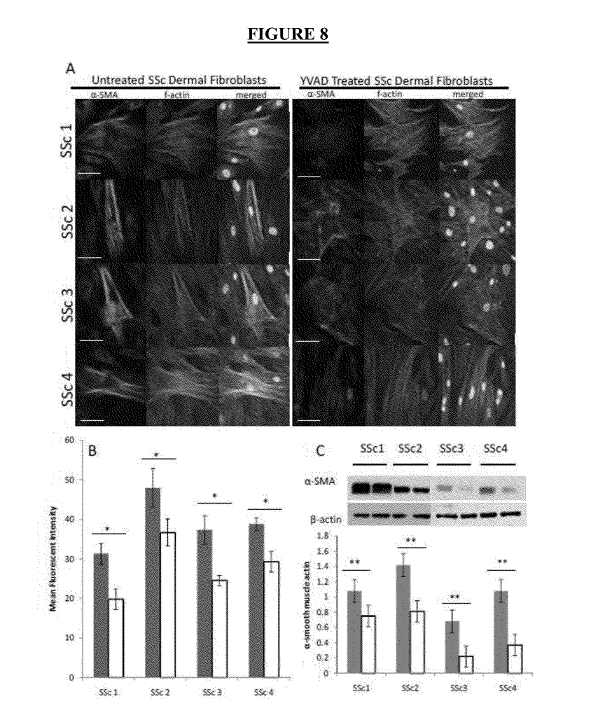

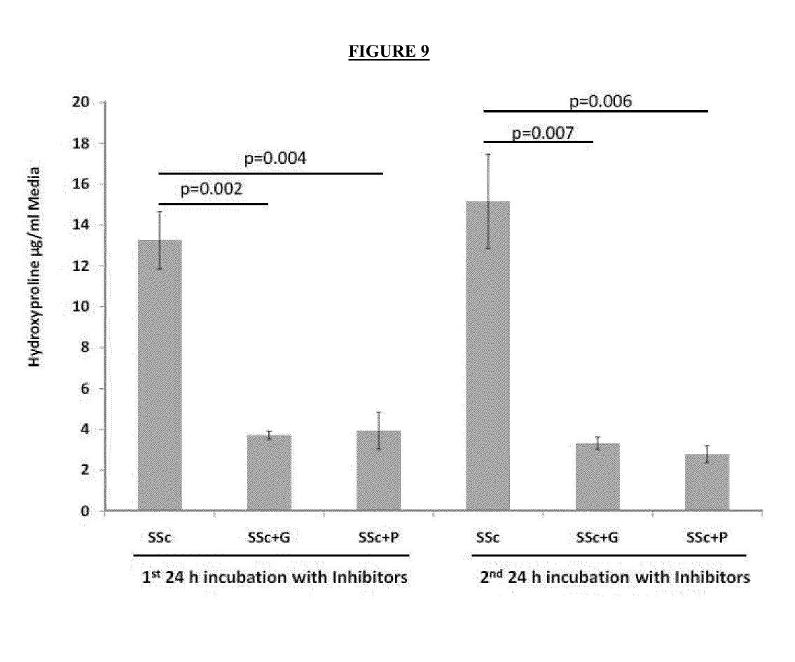

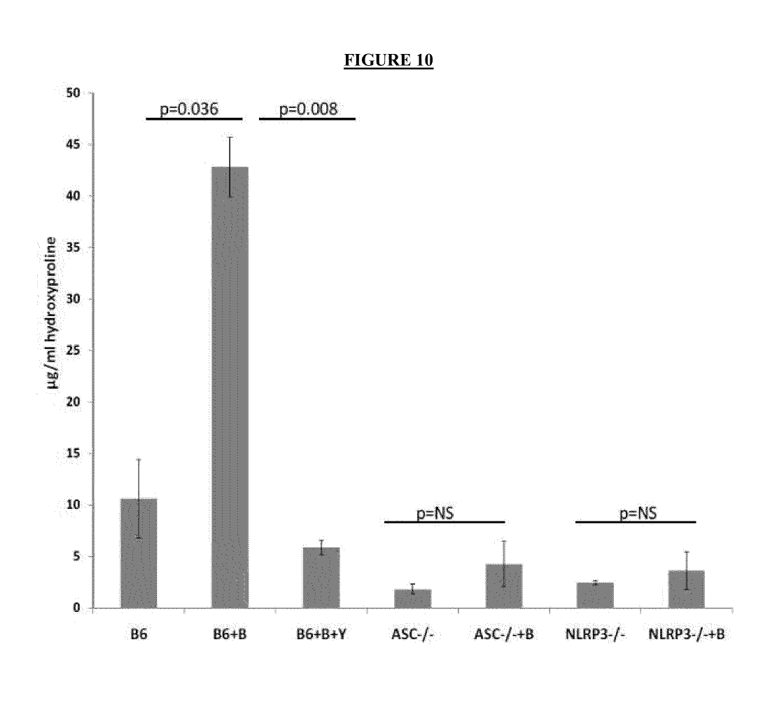

Direct inhibition of CASP1 abrogated collagen secretion and α-SMA expression (FIGS. 8 and 10). In one embodiment, this experimental finding is translated into a therapy for SSc. The efficacy of three selected compounds that inhibit different aspects of inflammasome signaling is determined. Anakinra blocks the IL-1 receptor. Glyburide inhibits NLRP3 activation by decreasing purinergic receptor activation and parthenolide inhibits multiple inflammasomes and NF-κB signaling. Glyburide and parthenolide are herein shown to be efficacious in new in vitro studies on SSc fibroblasts, with 70% decrease in collagen expression by 24 h treatment (FIG. 9). Effectiveness of these compounds at abrogating collagen deposition in vivo is studied in a mouse model of SSc. In one embodiment, parthenolide, glyburide, and/or anakinra aid in the resolution of already established fibrosis.

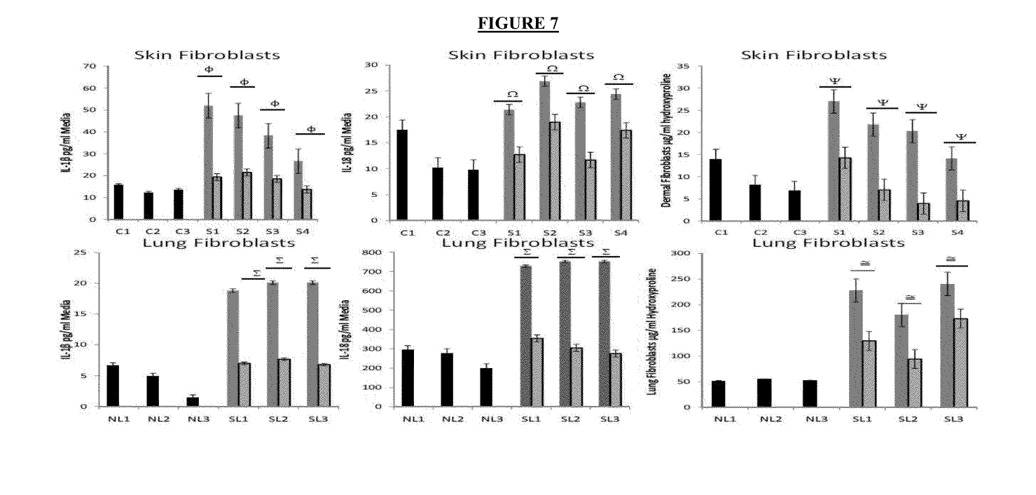

The present studies implicate for the first time the activation of the innate immune system mediated by the inflammasome as the driving force behind SSc dermal and lung fibrosis. Increased expression of AIM2, NLRP3, and NOD2 inflammasomes was observed in SSc fibroblasts. Further, inhibition of CASP1 activity significantly reduced collagen secretion (FIG. 7), and significantly decreased IL-1β and IL-18 secretion (FIG. 7). Furthermore, with inhibition of CASP1, α-SMA protein was decreased and the myofibroblast phenotype ameliorated (FIG. 8). These data suggest that active CASP1 mediated by inflammasome signaling drives the profibrotic phenotype in SSc. The experiments described herein allow one to determine why AIM2, NLRP3, and NOD2 inflammasomes are active and determine if autocrine signaling with IL-1β and IL-18 maintains a chronically activated inflammasome contributing to fibrosis. To date there are few effective therapeutics to abrogate SSc pathology, therefore investigating already existing inhibitors of the inflammasome and/or products is beneficial for the treatment of SSc.

Determining Whether Elevated Expression of AIM2, NLRP3 and NOD2 Inflammasomes is Found in Lung Myofibroblasts and Assessing Whether Inhibition of CASP4 and/or CASP5 Abrogates Secretion of IL-1β, IL-18 and Collagen

Gene expression analysis on SSc dermal fibroblasts indicated significant increases in three inflammasomes (AIM2, NLRP3, and NOD2), their adaptor molecules, and downstream signaling components. Therefore, experiments are performed to determine whether the same inflammasomes are upregulated in SSc lung fibroblasts, and in normal fibroblasts stimulated with bleomycin. Further, the specificity of inflammasome expression for myofibroblasts and the contribution of inflammatory caspases in myofibroblast activation are assessed.

Extension of the Array Data to Additional SSc Dermal Fibroblast Lines, SSc Lung Fibroblast Lines, and Normal Dermal Fibroblasts Stimulated with Bleomycin:

Experiments are performed with additional 12 SSc dermal cell lines and 12 normal dermal fibroblast cell lines. Furthermore, the inflammasome array in 15 lung fibroblast cell lines compared to 15 normal lung fibroblast cell lines is investigated. Comparisons between array expression in dermal fibroblasts and lung fibroblasts are made. All cell lines used comprise of early passage number (passage 2-4). Expression of inflammasome genes and downstream signaling molecules is investigated using the arrays in bleomycin stimulated normal dermal fibroblasts.

In Vitro Analyses of Inflammasome Transcripts and Inflammasome Mediated Protein Products in Myofibroblasts Vs. Fibroblasts from the Same Subject:

In order to define whether myofibroblasts are expressing higher levels of AIM2, NLRP3 and NOD2 transcripts than fibroblasts, primary cell cultures of myofibroblasts/fibroblasts isolated from skin and lung lesions (passage 2-4) are sorted for myofibroblasts using flow cytometry by selecting for OB-cadherin, a novel myofibroblast cell surface marker. The isolated myofibroblasts are assayed by RT-PCR for the inflammasome array and compared to fibroblasts from the same subject. It is then determined whether increased transcripts of AIM2, NLRP3 and NOD2 translates to increased protein expression in the cytoplasm of myofibroblasts vs. fibroblasts. The expression of these proteins in myofibroblasts is compared to the expression in fibroblasts from the same individual, to normal fibroblasts and normal fibroblasts treated with TGF-β1 for 5 days to induce the myofibroblast phenotype.

Because NOD2 associates with its adaptor molecule RIPK2 and XIAP when activated, it is determined whether NOD2 has assembled with RIPK2/XAIP. Co-immunoprecipitation for NOD2 is performed, and then the isolated protein for RIPK2 or XIAP is probed by Western blotting. Likewise, for NLRP3 and AIM2 co-immunoprecipitation and probe for PYD and PYDC1 are used, as these are their adaptor molecules that enable the inflammasomes to bind to ASC and activate CASP1.

Inflammasome and Caspase Knockdown:

Confirmation of inflammasome activation is determined using commercially available lentiviral siRNA for AIM2, NLRP3 and NOD2 (Santa Cruz Biotechnology, Santa Cruz Calif.) each on their own and then in combination. Once knocked down, CASP1 activity and the secretion of IL-1β, IL-18 and hydroxyproline are examined. The myofibroblast phenotype is also investigated to determine if myofibroblast differentiation is driven by one or more of these inflammasomes. In the arrays increased transcripts of the inflammatory caspases CASP1 (5.7 fold, p=0.007), CASP4 (2.7 fold, p=0.0015), and CASP5 (7.9 fold, p=0.043) were observed. As CASP1 contributes to inflammation, along with CASP4 and CASP5; and CASP4 and CASP5 can also mediate the cleavage of pro-IL-1β and pro-IL-18, the activity of CASP4 and CASP5 is investigated by ELISA or Western blotting. CASP5 was upregulated in lesional skin from psoriatic patients, suggesting that CASP5 may play an important role in the inflammatory response in skin. Z-WEHD-FMK inhibits CASP5 activity and Z-LEVDFMK inhibits CASP4 activity. These two new inhibitors are compared to the Z-YVAD-FMK inhibitor in the preliminary data (FIGS. 6-8) to inhibit CASP1 activity in the cell lines. The contribution of each caspase to IL-1β, IL-18 and hydroxyproline secretion is assessed by employing these inhibitors targeted specifically for each caspase alone and in combination.

Understanding the Mechanism of Inflammasome Activation and Downstream Signaling Initiated by the Pore Forming Complex P2X₇

SSc fibrosis appears to be mediated by CASP1 activation leading to IL-1β, IL-18 and collagen secretion, but the initiating event in this pathway is unknown. This investigation addresses P2X₇ engagement as a primary event to initiate IL-1β and IL-18 signaling and the downstream effects on TGFβ1 expression, as well as whether TGFβ1, and IL-1β and/or IL-18 cross signal to mediate fibrosis and maintain inflammasome activation. These studies also help determine whether TGFβ1 is a necessary intermediary for fibrosis to occur.

Analysis of the Activation of the Purinergic Receptor P2X7 and Pannexin-1 in SSc Fibroblasts and Myofibroblasts Isolated from SSc Patients:

The inflammasome in SSc fibroblasts is chronically activated as there is maintenance of increased inflammasome signatures in fibroblasts that have been passaged numerous times (data not shown). ATP is the main endogenous ligand that activates the purinergic receptor P2X₇, leading to the rapid opening of a potassium-selective channel followed by the gradual opening of a larger pore that is mediated by pannexin-1, Activation of the purinergic receptor results in potassium efflux, plasma membrane depolarization, cell swelling, disaggregation of the cytoskeletal network, leading to inflammasome activation and IL-1β and IL-18 release. In confirmation of this finding, P2X₇ deficient mice are defective IL-1β release by ATP. The expression and downstream signaling by P2X₇ and pannexin-1 proteins in SSc (myofibroblasts vs. fibroblasts) is investigated using P2X₇ and pannexin-1 antagonists. P2X₇ signals to activate CASP1 via the inflammasome leading to the subsequent processing and release of IL-1β. Further downstream signaling by the P2X₇ receptor induces the secretion of IL-6 and the activation of ERK1/2 and p38 leading to increased MCP-1, proteins that are known to be elevated in SSc fibroblasts. In the array data, 1.9 fold increases in P2X₇ were observed, suggesting that increased P2X₇ may play a role in SSc pathology and that myofibroblasts may have higher expression of P2X₇ than fibroblasts.

P2X₇ may be a critical switch for CASP1 activation and inflammasome formation. The signaling by ATP in SSc fibroblasts vs. myofibroblasts is thus elucidated and its downstream effects via the purinergic receptor P2X₇ and pannexin-1 are determined. SSc fibroblasts and SSc myofibroblasts are collected (sorted by flow cytometry as described elsewhere herein) and the release of IL-1β, IL-18 and hydroxyproline is measured, along with CASP1 activity when stimulated with 50 μM ATP, 0.6 μM BzATP, or 10 μM oATP. These results are compared to normal fibroblasts treated with TGF-β1 for 5 days. However, stimulation with ATP alone may not be enough to activate CASP1; therefore LPS may be added as an additional stimulus. Once activated, P2X receptors trigger increased matrix deposition, thus the secretion of hydroxyproline for total collagen in the cells when stimulated with BzATP, oATP, ATP, (and LPS if needed) is measured and compared with unstimulated fibroblasts. Because IL-6 release can also be mediated by P2X₇ stimulation in SSc fibroblasts, IL-6 release by SSc fibroblasts and myofibroblasts is investigated with ATP, BzATP and oATP, comparing it to normal cells and fibroblasts stimulated with TGF-β1 for 5 days by ELISA.

The pannexin-1 hemichannel forms a non-selective pore for molecules sized <1 kD. Uptake of the fluorescent dye YoPro-1 (Invitrogen) can occur within seconds to minutes after P2X₇ receptor stimulation. The P2X₇ receptor can be inhibited with KN-62 (1-[N,O-bis(5-isoquinolinylsulfonyl)-N-methyl-L-tyrosyl]-4-phenylpiperazine) to prevent ATP induced YoPro-1 uptake but fails to inhibit other P2X or P2Y receptors. Pannexin-1 is essential for NLRP3 inflammasome activation leading to CASP1 maturation and IL-1β secretion. The activation of pannexin-1 in SSc fibroblasts and myofibroblasts is studied and compared to normal fibroblasts and fibroblasts stimulated for 5 days with TGF-β1. Pannexin-1 activation has been demonstrated by the uptake of 1 μM YO-PRO dye into cells during over time. The initial time studies starts at 1 min. A difference between SSc (fibroblasts vs. myofibroblasts) with normal fibroblasts and fibroblasts stimulated for 5 days with TGF-β1 may be observed. However 1 min may not be sufficient time to observe differences and therefore the timing of this experiment may have to be adjusted. Confirmation of the involvement of P2X₇ and pannexin-1 is determined by lentiviral siRNA knockdown of these genes in SSc cells (Santa Cruz Biotechnology). Pannexin-1 may also be inactivated with the pannexin-1 mimetic peptide WRQAAFVDSY or carbenoxolone.

Investigation of the Mechanism(s) of Activation of the Inflammasome that Mediate the Expression of the Profibrotic Cytokine TGFβ1 and to Determine if Autocrine Signaling Maintains the Activation of the Inflammasome in SSc Cells:

PPARγ is important for the regulation of collagen expression and induced PPARγ expression abrogates fibrosis (Ghosh et al., 2009, FASEB J 23:2968-77). SSc fibroblasts can induce large quantities of type I interferon, which has a role in fibrosis (Farina et al., 2010, J Invest dermat 130:2583-93), and furthermore AIM2 is one inflammasome that upregulates the expression of type I interferons (Barber, 2011, Curr Opin Immunol). Indeed, in the gene arrays studies SSc fibroblasts had a 6.8 fold increase in INF-β1 when compared with normal fibroblasts (p=0.023). This study further investigates IL-1β and IL-18 signaling and its downstream effects on TGF-β1 expression; and determines if TGF-β1, IL-1β and/or IL-18 cross signal to mediate fibrosis by maintaining the activation of the AIM2, NLRP3 and/or NOD2 inflammasomes. Myofibroblasts are the prime cell in the SSc lesion that expresses the high amounts of collagen. These cells may be considered part of the innate immune system as they display many functions and receptors common to macrophages; and inhibition of CASP1 abrogated the myofibroblast phenotype by reducing α-SMA protein expression (FIG. 8).

These studies focus on signaling derived from stimulated AIM2, NLRP3, and NOD2 inflammasomes and determining their role in collagen secretion. Normal fibroblasts are stimulated with ATP to activate the NLRP3 inflammasome, double-stranded DNA to activate AIM2, and peptidoglycan to activate NOD2, If necessary, LPS is added as a second stimulus. Observation of increased secretion of IL-1β and IL-18 produced by AIM2, NLRP3 and NOD2 is expected. Secretion of IL-1β and IL-18 is measured by ELISA, when each inflammasome is stimulated alone, or in combination by ELISA. In the stimulated cells, the expression of inflammasomes and downstream inflammasome mediated products is investigated by the inflammasome array (SABiosciences). This allow one to not only confirm the activation of the inflammasome by the chosen stimulus but also to examine any differences observed between the inflammasome already observed in SSc fibroblasts vs. a single activated inflammasome. In addition, the contribution of each specific inflammasome to the downstream signaling molecules such as CCL5, CCL7, IL-6, IL-33, and IFN-β1 is identified, as all of these molecules were significantly upregulated in the SSc arrays. As TGF-β1 is a significant cytokine inducing fibrosis in SSc, the expression of TGF-β1 when each of these inflammasomes is activated (mRNA and active protein) is investigated. Because these downstream signaling molecules are most likely driven by autocrine signaling from IL1β and/or IL-18 antibodies against IL-1β and IL-18 (each alone and together) are used to inhibit autocrine signaling, and conversely, autocrine signaling will be enhanced with the addition of recombinant IL-1β and IL-18 (each alone and together). Collagen secretion by hydroxyproline, and PPARγ, active TGF-β1, α-SMA, AIM2, NLRP3 and NOD2 expression is measured by Western blotting. These results are compared to fibroblasts stimulated with bleomycin, known to activate the inflammasome (FIG. 10).

The information gleaned here allows one to to determine if fibroblasts need a single stimulatory event like monocytes, or two distinct stimuli like macrophages. In addition, these studies identify feedback mechanisms that maintain inflammasome signaling mediated by downstream products such as TGF-β1. The findings above are further investigated in NLRP3−/− and ASC−/− fibroblasts and the above studies in these knockouts are complemented by the addition of TGF-β1. This allows one to directly compare the contribution of the inflammasome products to fibrosis and determine if TGF-β1 signaling requires a functional inflammasome for the establishment of fibrosis. Control fibroblasts are C57BL/6 fibroblasts as this is the genetic background for the knockout animals.

Investigation of Activation of NF-κB Via NOD2 and IL-1 Receptor Signaling.

The NOD2 inflammasome activates NF-κB (FIG. 6) and NF-κB activation can induce the transcription and translation of IL-1β and IL-18 to their immature form. NOD2 upregulates transcription of IFN-β1. There were increased transcripts of the NOD2 inflammasome in SSc fibroblasts. IL-1β can activate NF-κB. These studies determine the activity of NF-κB in SSc fibroblasts.

The classical NF-κB pathway is investigated by exploring the expression of p65 and phosphorylation of IKBα, and the alternate pathway that encompasses RelB and p52 by Western blot to determine if NF-κB activity is elevated. It is determined if NOD2 and/or IL-1β are modulating NF-κB activity by employing lentiviral siRNA for NOD2 and the IL-1 receptor (Santa Cruz Biotechnology). These studies are followed by blocking signaling from the IL-1 receptor using antibodies.

### Investigating Inflammasome Inhibitors as Potential Therapeutics to Downregulate SSc Inflammation and Fibrosis.

These studies utilize the FDA approved drugs anakinra, glyburide, and parthenolide. These drugs inhibit the inflammasome activation or inhibit downstream signaling byproducts of the inflammasome. Assays are performed in vitro in SSc fibroblasts. Also utilized is the bleomycin mouse model of SSc previously used to conclusively demonstrate that bleomycin activates the NLRP3 inflammasome. Fibrosis is induced with bleomycin, and the mice are treated with the inflammasome inhibiting drugs. Dermal thickness, serum cytokine levels, and inflammatory cell infiltrates are assessed.

Analyses with inhibitors of the inflammasome or downstream inflammasome products are performed, to further determine the role of the inflammasome in SSc fibrosis and to ascertain if one or more of these inhibitors may be efficacious in the treatment of this disease. These studies are performed in mice.

Anakinra (Kineret) is an IL-1 receptor antagonist manufactured by Amgen. It has been shown to be effective in treating many diseases mediated by IL-1β including rheumatoid arthritis. Anakinra is purchased from Amgen.

Glyburide (glibenclamide) has been used to treat type 2 diabetes. The drug also inhibits the NLRP3 inflammasome by inhibiting ATP-sensitive potassium channels and inducing membrane depolarization. The depolarization in the membrane induces opening of voltage-dependent calcium channels, causing an increase in intracellular calcium and potassium. Glyburide is purchased from Sigma.

Parthenolide inhibits the activation of multiple inflammasomes preventing CASP1 activation and NFκB signaling. Parthenolide is purchased from Sigma.

Further understanding of the role of the inflammasome in SSc fibrosis is obtained using the inflammasome signaling inhibitors, anakinra, glyburide, and parthenolide. These inhibitors affect different parts of the inflammasome; either by directly inhibiting activation of the NLRP3 inflammasome, inflammasome assembly, or downstream signaling components including CASP1 activation and IL-1.

The initial studies are performed in vitro on SSc myofibroblasts to determine the efficacy of the inhibitors anakinra (5 μM), glyburide (100 μM), and parthenolide (1 μM). The inhibitors are investigated during a time course and the initial changes in inflammasome signaling are determined, and then subsequent differences in the expression of molecules that are induced by downstream signaling from IL-1β and IL-18 are investigated. For example, later cytokines that can be dependent on the expression of IL-1β are IL6 and TGFβ1. In one embodiment, a decrease in these cytokines is not observed within 60 min but is observed within 24 or 48 h. ECM production is measured including COL1A1, COL3A1. Furthermore, as increase in NLRP3 protein and its downstream signaling products, CASP1 activation, IL-1β, and IL-18 were observed in SSc, these proteins are measured with the inhibitors. In one embodiment, inflammasome signaling products are inhibited without any detriment to the cell such as the induction of apoptosis. Therefore, the activation of CASP3 is investigated as a measure of early apoptotic events.

For in vivo analyses, the efficacy of the inflammasome inhibitors in the bleomycin mouse model of fibrosis is investigated. Bleomycin signals through the NLRP3 inflammasome (FIG. 8). Specific pathogen free C57Bl/6 mice are purchased from Jackson Laboratories (Bar Harbor, Me.). Dermal fibrosis is induced by injecting 100 μl of bleomycin (1 mg/ml) daily for 28 days. As a control, the contralateral flank of the same mouse is injected with 100 μl of sterilized PBS. At the same time as the bleomycin and PBS injections, the mice are treated once a day with anakinra (3 mg/kg), parthenolide (5 mg/kg), or glyburide (5 mg/kg). Untreated mice serve as a control.

Skin tissue from both injection sites is harvested along with the right lobe of the lung, and the tissues fixed in 4% paraformaldehyde, embedded in paraffin, and sectioned onto positively charged slides. Sections are stained with Masson's Trichrome for collagen content and photographed using a Nikon Eclipse 80i Microscope and Advanced SpotCam software for the digital camera and skin thickness measured (data not shown). Dermal thickness is measured as the distance from just below the epidermis to the muscle fascia layer and compared to the dermal thickness at the PBS injection site and non-injected animals.

Moreover, to determine if the recited compositions can reverse already established fibrosis and in an additional study after 28 days of bleomycin injections, the mice are treated with anakinra, parthenolide, or glyburide (described above), and the resolution of fibrosis at the injection site and in the lung is followed every 7 days as described above. Bleomycin fibrosis resolves in approximately 60 days once the injections have ceased.

In one embodiment, Bay 11-7082 is useful within the methods of the invention. BAY 11-7082 inhibits inflammasome assembly and NFκB activation. In another embodiment, colchicine, originally isolated from the plant Colchicum autumnale, inhibits CASP1 activation. Colchicine has been used to treat gout that is mediated by the NLRP3 inflammasome. Colchicine can be toxic at high levels and some individuals are intolerant of it, however in combination with Anakinra it appears to be more efficacious. The invention contemplates combinations of compositions for the treatment of fibrosis.

### Inflammasome Components and Downstream Signaling Molecules are Upregulated in SSc Dermal Fibroblasts.

84 genes array involved in the function of inflammasome signaling and the innate immune response (SABiosciences, Frederick Md.) were investigated by RT-PCR in SSc dermal fibroblasts (n=3) compared to normal dermal fibroblasts (n=3). These lines were age and sex matched, in addition to undergoing the same number of passages (4-6) in culture. Forty genes were found to have more than a 2 fold increase in expression in SSc fibroblasts over normal fibroblasts. Selected genes important to this application are reported in Table 1. These results suggest that inflammasomes and downstream signaling molecules are elevated in SSc dermal fibroblasts. RT-PCR inflammasome array thus revealed increased expression of 3 distinct inflammasomes, their accessory proteins, and enhanced expression of downstream signaling molecules. It is thus novel that activation of inflammasome signaling molecules was found in fibroblasts, and that active CASP1 drives the myofibroblast phenotype (FIG. 7).

TABLE 1
Selected genes in the inflammasome array found
to have >2-fold increase over normal fibroblasts.

| Gene | Fold increase | p value |
|---|---|---|
| AIM2 | 11.8 | 0.017 |
| NLRP3 | 7.0 | 0.028 |
| NOD2 | 6.1 | 0.0045 |
| CARD6 | 13.7 | 0.00002 |
| RIPK2 | 2.3 | 0.0089 |
| XIAP | 2.3 | 0.05 |
| Pyrin | 6.9 | 0.035 |
| PYDC1 | 8.3 | 0.042 |
| CASP1 | 5.7 | 0.007 |
| CASP4 | 2.7 | 0.0015 |
| CASP5 | 7.9 | 0.043 |
| CCL5 | 8.3 | 0.026 |
| CCL7 | 4.2 | 0.032 |
| IL-6 | 8.7 | 0.0047 |
| IL-33 | 4.1 | 0.025 |
| INF-β1 | 6.8 | 0.023 |
| IL-1β | 7.1 | 0.020 |
| IL-18 | 5.0 | 0.0019 |

Key: Red=inflammasomes, orange=accessory molecules to the three inflammasomes; green=inflammatory caspases; pink=inflammatory cytokines.

Expression of AIM2, NLRP3, and NOD2 inflammasome proteins may be assessed in SSc dermal and lung fibroblasts. AIM2 and NLRP3 activate CASP1 and inhibition of CASP1 abrogated collagen secretion (FIG. 6). NOD2 and its adaptor proteins RIPK2 and XIAP play a role in autoimmunity. NLRP3 is a sensor for cellular stress and has been found to be activated by a plethora of stimuli. AIM2 regulates the expression of type I interferon.

In one embodiment, fibroblast cell lines established from diffuse SSc are studied. SSc patients may have different gene signatures based on the array data published by the Whitfield group (Sargent et al., 2010, J Invest Dermatol 130:694-705). Diffuse SSc may thus be divided into 2 subsets; TGF-β activated vs. TGF-β not activated based on the expression of E2F7, GDF6 and ACTA. The RT-PCR inflammasome array data may be expanded to a larger cohort and also used to investigate inflammasome expression in lung fibroblasts.

### Inhibition of Inflammasome Mediated CASP1 Activity Downregulates IL-1β, IL-18, and Collagen Secretion (as Detected by Hydroxyproline Assay) in SSc Fibroblasts.

Z-YVAD(OMe)-FMK (YVAD) inactivates CASP1 activity. Thus, IL-1β, IL-18 were investigated by ELISA, as well as collagen secretion (using a standard assay for hydroxyproline) by SSc fibroblasts when treated with YVAD. SSc dermal fibroblasts (n=4) exhibited increased IL-1β secretion compared to normal dermal fibroblasts (n=3). Normal dermal fibroblasts secreted 13.6±0.7 pg/ml of IL-1β. With the addition of YVAD to SSc dermal fibroblasts the secretion of IL-1β was reduced from 41.1±5.6 pg/ml to 12.0 pg/ml (p=0.012, FIG. 7), and this was similar to levels secreted by normal dermal fibroblasts (p=0.08). Normal lung fibroblasts (n=3) secreted 3.3±0.5 pg/ml of IL-1β. YVAD reduced the secretion of IL-1β by SSc lung fibroblasts (n=3) from 19.7±0.4 pg/ml to 7.2±0.2 pg/ml (p=0.001, FIG. 7), and this was also similar to that secreted by non-fibrotic lung fibroblasts (p=0.2).

IL-18 secreted by normal dermal fibroblasts was 12.4±2.0 pg/ml. With the addition of YVAD to SSc dermal fibroblasts, IL-18 was reduced from 23.9±1.0 pg/ml to 15.2±1.5 pg/ml (p=0.008, FIG. 7), and was not statistically different from normal dermal fibroblasts (p=0.41). Normal lung fibroblasts secreted 256.8±23.5 pg/ml of IL-18. The addition of YVAD to SSc lung fibroblasts reduced IL-18 secretion from 743.9±6.4 pg/ml to 311.6±18.5 pg/ml (p=0.001, FIG. 7), a level that was not statistically different from normal lung fibroblasts (p=0.2). These results indicate that caspase-1 activation in SSc fibroblasts is required for IL-1β and IL-18 secretion.

IL-1β or IL-18 can enhance collagen expression in fibroblasts and suppression of these cytokines abrogates collagen secretion, therefore it was investigated if inhibiting CASP1 activity would also reduce collagen expression. Hydroxyproline was used as a measure of total collagen secreted into the culture media. Normal dermal fibroblasts secreted 9.7±2.19 μg/ml of hydroxyproline. SSc dermal fibroblasts treated with YVAD decreased the secretion of hydroxyproline from 18.73±2.33 μg/ml to 5.19±0.94 μg/ml (p=0.003; FIG. 7), and this was found to be similar to hydroxyproline secretion by normal dermal fibroblasts (p=0.5). Likewise in SSc lung fibroblasts, inhibition of CASP1 with YVAD decreased total collagen secretion. Normal lung fibroblasts secreted 52.2±1.0 μg/ml hydroxyproline. SSc lung fibroblasts treated with YVAD decreased the secretion of hydroxyproline from 216.1±18.5 μg/ml to 132.0±22.8 μg/ml (p=0.011; FIG. 7) and this was found to be similar to hydroxyproline secretion by normal lung fibroblasts (p=0.07). These results indicate that CASP1 activation is required for the elevated collagen secretion observed in SSc fibroblasts. In summary, relative to healthy fibroblasts, SSc fibroblasts had increased levels of IL-1β, IL-18 and hydroxyproline secretion. Inhibition of CASP1 activity abrogated this increase.

### Inhibition of CASP1 Decreases α-Smooth Muscle Actin and Reduces Myofibroblast Morphology.

Myofibroblasts secrete excessive levels of collagen in the skin and internal organs in SSc and these cells have a distinct morphology containing discrete α-smooth muscle actin (α-SMA) fibers. Because there was less collagen, IL-1β, and IL-18 secreted by SSc cells treated with YVAD, it was determined if α-SMA, a marker for myofibroblasts, was also reduced. The morphology of SSc myofibroblasts was investigated when stained for α-SMA (green) and f-actin (red). Untreated SSc dermal myofibroblasts presented with strongly staining α-SMA and thickened contractile fibers (FIG. 8A). After 48 h treatment with YVAD, the α-SMA expression was decreased and the contractile fibers became less pronounced (FIG. 8A). Mean fluorescent intensity of α-SMA by SSc dermal myofibroblasts was 38.9±2.4 and this declined to 27.6±2.5 (p=0.0005; FIG. 8B) with the CASP1 inhibitor. F-actin staining was unaffected by YVAD treatment and the cytoskeleton of the myofibroblasts (FIG. 8A) and fibroblasts (not shown) was as pronounced in the treated and untreated cells. Western blotting was performed to quantify α-SMA expression in SSc dermal fibroblasts (FIG. 8C). α-Smooth muscle actin was decreased approximately 50% in the YVAD treated SSc fibroblasts after normalization for β-actin loading (1.07±0.15 in untreated vs. 0.54±0.14 in treated cells, p=0.008). These results suggest that activation of CASP1 is required for myofibroblast differentiation. In summary, myofibroblast differentiation was dependent on downstream inflammasome signaling that promotes CASP1 activity as inhibition of CASP1 activity ameliorated α-SMA protein and the myofibroblast phenotype.

Collagen Secretion by SSc Fibroblasts can be Abrogated with Glyburide or Parthenolide.

Because the model is that inflammasomes are activated in SSc, it was tested whether if inhibition of the inflammasomes with the compositions recited herein could reduce collagen secretion. SSc fibroblasts were treated for 24 h with 100 μM Glyburide or 10 μM Parthenolide. The media was replaced with fresh after 24 h with 100 μM Glyburide or 10 μM Parthenolide and cultured for an additional 24 h. The culture media was assayed for hydroxyproline. By 24 h, collagen secretion was reduced by approximately 70% with Glyburide and Parthenolide (FIG. 9, p=0.002 and p=0.004 respectively and with 48 h treatment p=0.007 and p=0.006 respectively).

These studies are used to investigate whether a composition inhibits the inflammasome signaling directly or inhibits products of the inflammasome. Assays are performed in vitro on SSc fibroblasts and in vivo utilizing mice and bleomycin. Bleomycin signals through the inflammasome to activate CASP1 and promote collagen secretion (FIG. 9).

### The Inflammasome Participates in Bleomycin Induced Dermal Fibrosis.

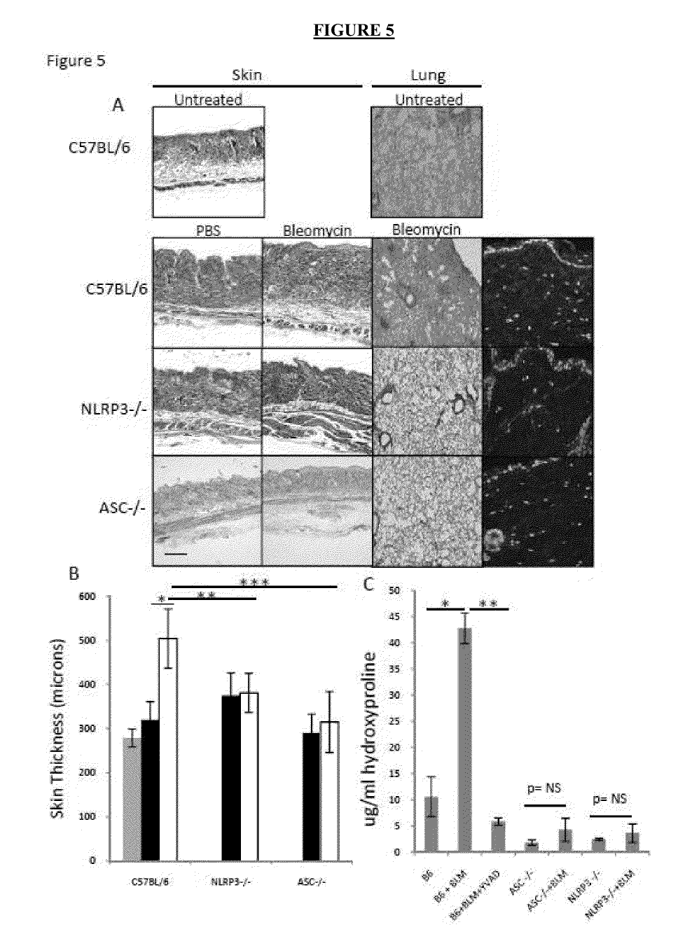

The role of the inflammasome proteins in dermal fibrosis in vivo was determined by utilizing the murine bleomycin-induced model of fibrosis. Using the model both the NLRP3 and ASC proteins were found to be absolutely necessary for the development of dermal fibrosis. As expected C57Bl/6 mice had increased collagen deposition with marked skin thickening induced by bleomycin. In the PBS injected skin, average skin thickness was 320.7±40.6 microns whereas in the bleomycin injected skin it was 504.4±66.7 microns (1.54±0.11 fold increase in thickness; p=0.043, not shown). The bleomycin induction of skin thickness was abolished in the skin of NLRP3−/− (1.10 fold induction compared to 1.54 fold in C57BL/6 mice; p=0.002; not shown) and ASC−/− (1.06 fold induction compared to 1.54 fold induction in C57BL/6 mice; p=0.01; not shown). Fibroblasts established from the skin of the mice were stimulated with bleomycin and CASP1 activity blocked with YVAD. Secretion of hydroxyproline was measured (FIG. 9). C57BL/6 fibroblasts secreted 10.6±3.8 μg/ml hydroxyproline and when stimulated with 1 μM bleomycin secreted 42.8±2.9 μg/ml hydroxyproline (p=0.036). The secretion of hydroxyproline could be inhibited by YVAD (5.9±0.7 μg/ml hydroxyproline, p=0.008, FIG. 5). In ASC−/− and NLRP3−/− fibroblasts, hydroxyproline secretion was not induced by bleomycin (p>0.43; FIG. 10). These results suggest that the inflammasome and caspase-1 activation is required for bleomycin induced in vivo skin fibrosis and in vitro collagen secretion. In summary, bleomycin activates the inflammasome and deletion of NLRP3 or ASC abrogated the induced secretion of hydroxyproline in fibroblasts when stimulated with bleomycin. Inhibition of CASP1 abrogated secretion of hydroxyproline in fibroblasts stimulated with bleomycin.

### Antibodies

In one aspect, the invention contemplates using a composition comprising a caspase-1 antibody or an IL-1β antibody. In another embodiment, the antibody is selected from the group consisting of XOMA-052, AMG-108, canakinumab, rilonacept, LY-2189102, and any combinations thereof. In one embodiment, the antibody comprises an antibody selected from a polyclonal antibody, a monoclonal antibody, a humanized antibody, a synthetic antibody, a heavy chain antibody, a human antibody, and a biologically active fragment of an antibody.

It will be appreciated by one skilled in the art that an antibody comprises any immunoglobulin molecule, whether derived from natural sources or from recombinant sources, which is able to specifically bind to an epitope present on a target molecule. In one embodiment, the target molecule comprises caspase-1 or IL-1β.

In one aspect of the invention, the target molecule is directly neutralized by an antibody that specifically binds to an epitope on the target molecule. In another aspect of the invention, the effects of the target molecule are blocked by an antibody that specifically binds to an epitope on a downstream effector. In still another aspect of the invention, the effects of the target molecule are blocked by an antibody that binds to an epitope of an upstream regulator of the target molecule.

When the antibody to the target molecule used in the compositions and methods of the invention is a polyclonal antibody (IgG), the antibody is generated by inoculating a suitable animal with a peptide comprising full length target protein, or a fragment thereof, an upstream regulator, or fragments thereof. These polypeptides, or fragments thereof, may be obtained by any methods known in the art, including chemical synthesis and biological synthesis, as described elsewhere herein. In this regard, an exemplary IL-1β sequence is SEQ ID NO:1. Antibodies produced in the inoculated animal that specifically bind to the target molecule, or fragments thereof, are then isolated from fluid obtained from the animal.

Antibodies may be generated in this manner in several non-human mammals such as, but not limited to goat, sheep, horse, camel, rabbit, and donkey. Methods for generating polyclonal antibodies are well known in the art and are described, for example in Harlow et al., 1998, In: Antibodies, A Laboratory Manual, Cold Spring Harbor, NY.

Monoclonal antibodies directed against a full length target molecule, or fragments thereof, may be prepared using any well-known monoclonal antibody preparation procedures, such as those described, for example, in Harlow et al. (1998, In: Antibodies, A Laboratory Manual, Cold Spring Harbor, N.Y.) and in Tuszynski et al. (1988, Blood, 72:109-115). Human monoclonal antibodies may be prepared by the method described in U.S. Patent Publication No. 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al., 1992, Critical Rev. Immunol. 12(3,4):125-168, and the references cited therein.

When the antibody used in the methods of the invention is a biologically active antibody fragment or a synthetic antibody corresponding to antibody to a full length target molecule, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology available in the art, and described, for example, in Wright et al., 1992, Critical Rev. in Immunol. 12(3,4):125-168 and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen et al. (U.S. Pat. No. 6,180,370), Wright et al., 1992, Critical Rev. Immunol. 12(3,4):125-168, and in the references cited therein, or in Gu et al., 1997, Thrombosis & Hematocyst 77(4):755-759, or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well-known procedures. Preferably, the human constant region DNA sequences are isolated from immortalized B-cells as described in International Patent Application Publication No. WO 198702671. CDRs useful in producing the antibodies of the present invention may be similarly derived from DNA encoding monoclonal antibodies capable of binding to the target molecule. Such humanized antibodies may be generated using well-known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., 1998, Antibodies: A Laboratory Manual, Cold Spring Harbor, N.Y.

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al., 1989, Nature 341:544-546 (incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as target molecule depletors in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA that specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., 2001, Molecular Cloning: A Laboratory Manual, Cold Spring Harbor Laboratory Press, Cold Spring Harbor, N.Y.

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell which expresses the corresponding antigen, the bacteriophage will bind to the cell. Bacteriophage that do not express the antibody will not bind to the cell. Such panning techniques are well known in the art and are described for example, in Wright et al., 1992, Critical Rev. Immunol. 12(3,4):125-168.

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage that display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the invention, or to prepare a pharmaceutical composition useful in practicing the method of the invention.

The antibodies of the present invention can be assayed for immunospecific binding by any method known in the art. The immunoassays that can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Current Protocols in Molecular Biology, (Ausubel et al., eds.), Greene Publishing Associates and Wiley-Interscience, New York (2002)). Exemplary immunoassays are described briefly below (but are not intended to be in any way limiting).

### Methods of the Invention

The invention includes a method of treating or preventing fibrosis in a subject afflicted with scleroderma. The method comprises administering to the subject a therapeutically effective amount of a pharmaceutical composition comprising an agent that inhibits formation of at least one inflammasome signaling product in the subject, whereby the fibrosis in the subject is treated or prevented.

In one embodiment, the agent that inhibits formation of at least one inflammasome signaling product is selected from the group consisting of glyburide, parthenolide, BAY 11-7082, colchicine, a caspase-1 inhibitor, an IL-1β-depleting agent, and any combinations thereof. In another embodiment, the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, a ribozyme, an antisense, an aptamer, a peptidomimetic, a small molecule, and any combinations thereof. In yet another embodiment, the IL 1β antibody comprises an antibody selected from the group consisting of a polyclonal antibody, monoclonal antibody, humanized antibody, synthetic antibody, heavy chain antibody, human antibody, biologically active fragment of an antibody, and any combinations thereof. In yet another embodiment, the caspase-1 inhibitor is selected from the group consisting of a caspase-1 antibody, siRNA, ribozyme, antisense, aptamer, peptidomimetic, small molecule, and a combination thereof. In yet another embodiment, the caspase-1 antibody comprises an antibody selected from the group consisting of a polyclonal antibody, monoclonal antibody, humanized antibody, synthetic antibody, heavy chain antibody, human antibody, biologically active fragment of an antibody, and any combinations thereof.

In one embodiment, the method further comprises administering to the subject an additional anti-scleroderma composition. In another embodiment, the anti-scleroderma composition comprises a vasodilator, an endothelin receptor antagonist, an angiotensin converting enzyme inhibitor, an immunosuppressive agent, halofuginone, CAT-192, cyclophosphamide and rabbit antithymocyte globulins, dasatinib, fludarabine, imatinib mesylate (Gleevec), nilotnib (Tasigna) or rituximab. In yet another embodiment, the agent and the anti-scleroderma composition are co-administered to the subject. In yet another embodiment, the agent and the anti-scleroderma composition are co-formulated and co-administered to the subject. In yet another embodiment, the composition in a pharmaceutically acceptable carrier is administered to the subject by an administration route selected from the group consisting of inhalational, oral, rectal, vaginal, parenteral, topical, transdermal, pulmonary, intranasal, buccal, ophthalmic, intrathecal, and any combination thereof. In yet another embodiment, the subject is a mammal. In yet another embodiment, the mammal is a human.

### Kits of the Invention

The invention includes a kit comprising an agent that inhibits formation of at least one inflammasome signaling product in a subject, an applicator, and an instructional material for use thereof. In one embodiment, the agent that inhibits formation of at least one inflammasome signaling product is selected from the group consisting of glyburide, parthenolide, BAY 11-7082, colchicine, a caspase-1 inhibitor, an IL-1β-depleting agent, and any combinations thereof. In another embodiment, the instructional material comprises instructions for preventing or treating fibrosis in a subject afflicted with scleroderma. In yet another embodiment, the instructional material recites that the agent is to be administered to the subject in an amount sufficient to inhibit formation of at least one inflammasome signaling product in the subject, whereby the fibrosis in the subject is treated or prevented.

In one embodiment, the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, ribozyme, an antisense, an aptamer, a peptidomimetic, a small molecule, and any combination thereof. In another embodiment, the caspase-1 inhibitor is selected from the group consisting of a caspase-1 antibody, siRNA, ribozyme, antisense, aptamer, peptidomimetic, small molecule, and a combination thereof. In yet another embodiment, the subject is a mammal. In yet another embodiment, the mammal is human.

### Combination Therapies

The compounds identified using the methods described here are useful in the methods of the invention in combination with at least one additional compound useful for treating scleroderma. This additional compound may comprise compounds identified herein or compounds, e.g., commercially available compounds, known to treat, prevent, or reduce the symptoms of fibrosis and/or scleroderma.

Non-limiting examples of anti-scleroderma agents considered within the invention include:

Vasodilators: Calcium channel blockers: nifedipine, Adalat (Bayer), Procardia; Prostaglandins: iloprost or epoprostenol (Flolan from GlaxoSmithKline)

Endothelin Receptor Antagonists: Prevent endothelin-mediated narrowing of blood vessels (such as Bosentan (Tracleer)).

Angiotensin Converting Enzyme Inhibitors (ACE): appear to be the most effective for scleroderma patients because of their protective actions in the kidney, including Capoten (captopril), enalapril (Vasotec), quinapril (Accupril)

Immunosuppressive Agents: Penicillamine

Halofuginone (Collgard Biopharmaceuticals): inhibitor of collagen type I synthesis; received Orphan drug status for scleroderma in 2000 and is currently in Phase II clinical trials. Halofuginone has the potential to affect the development of scleroderma but requires stoichiometric amounts to be effective.

CAT-192 (Cambridge Antibody Technology/Genzyme): antibody to TGF-β1, which is involved in the regulation of collagen synthesis. It received orphan status in the US and in Europe in early 2002 and is currently in Phase I/II trials in Europe.

Cyclophosphamide and rATG (rabbit antithymocyte globulins)

Dasatinib

Fludarabine and donor peripheral stem cell transplant

Imatinib mesylate (Gleevec)

Nilotnib (Tasigna)

Rituximab

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Pharmaceutical Compositions and Formulations

The invention envisions the use of a pharmaceutical composition comprising an agent that inhibits at least one inflammasome signaling product or a salt thereof within the methods of the invention.

Such a pharmaceutical composition comprises an agent that inhibits at least one inflammasome signaling product or a salt thereof, in a form suitable for administration to a subject, or the pharmaceutical composition may comprise an agent that inhibits at least one inflammasome signaling product or a salt thereof, and one or more pharmaceutically acceptable carriers, one or more additional ingredients, or some combination of these. The agent that inhibits at least one inflammasome signaling product may be present in the pharmaceutical composition in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions are formulated using one or more pharmaceutically acceptable excipients or carriers. In one embodiment, the pharmaceutical compositions comprise a therapeutically effective amount of an agent that inhibits at least one inflammasome signaling product and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one that comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of cancer in a patient.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing cancer in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system.

Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically- or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form. For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation. In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Human studies were approved by the Institutional Review Boards at Drexel University and the University of Pittsburgh. Animal studies were approved by the Institutional Animal Care and Use Committee at Drexel University. Fibroblasts derived from skin (n=4) from SSc patients (University of Pittsburgh) aged between 27-54 (3 female, 1 male) with a disease duration of 0.5-6 years who had diffuse disease. Scl70 auto-antibody was identified in 3/4 patients, the remaining patient did not have this information available. All satisfied the criteria for classification of SSc (Masi et al., “Preliminary criteria for the classification of systemic sclerosis (scleroderma) Subcommittee for scleroderma criteria of the American Rheumatism Association diagnostic and therapeutic criteria committee”, Arthritis Rheum 1980; 23:581-90) and had the diffuse cutaneous clinical subset of the disease as defined by LeRoy et al (LeRoy et al., 1988, J Rheumatol 15:202-5). Fibroblasts derived from lung (n=3) were obtained from SSc patients (University of Pittsburgh) with pulmonary fibrosis diagnosed with UIP/mild pulmonary hypertension, or UIP/NSIP fibrosis and all were undergoing a lung transplant (2 female, 1 male, aged 34-47 years, disease duration is unknown).

### Cell Culture:

Normal human dermal fibroblasts (n=4) were purchased from the Coriell Institute (Camden, N.J.) and normal lung fibroblasts (n=3) were obtained from the University of Pittsburgh. Normal and SSc dermal fibroblasts (750,000 cells/dish) were cultured in DMEM (Mediatech Inc., Manassas Va.) supplemented with 10% FBS (Mediatech) and 1% penicillin/streptomycin (Mediatech). Protein lysates were collected for western blotting and media was collected for ELISA and hydroxyproline assays. SSc fibroblasts were treated with 20 μM caspase-1 inhibitor, Benzyloxycarbonyl-Tyr-Val-Ala-Asp(OMe)-fluoromethylketone (Z-YVAD(OMe)-FMK; Enzo Life Sciences, Plymouth Meeting Pa.) for 48 h. Fibroblasts from NLRP3−/−, and ASC−/− and C57BL/6 mice (Jackson Laboratories, Bar Harbor Me.) were established from skin and were treated with 1 μM bleomycin and 20 μM Z-YVAD(OMe)-FMK for 48 h. Z-YVAD(OMe)-FMK did not interfere with cell proliferation or induce apoptosis in fibroblasts (p=0.22 and data not shown).

### Hydroxyproline Measurements:

Total collagen secreted into media was measured by hydroxyproline (Woessner, 1961, Arch Biochem Biophys 93:440-7). Absorbances were read in a spectrophotometer at 550 nm and the concentration of hydroxyproline was determined against a standard curve of hydroxyproline.

### Western Blotting:

30 μl of media was size fractionated on a 5% SDS polyacrylamide gel (Invitrogen, Carlsbad Calif.) and the proteins transferred to a PVDF membrane (Invitrogen). Non-specific binding sites were blocked and then probed with rabbit-anti-human COL1A1, COL3A1, or α-SMA (Santa Cruz Biotechnologies, Santa Cruz Calif.) overnight at 4° C. The membrane was washed and incubated with goat anti-rabbit-HRP (1:5000) (Jackson Immunoresearch, West Grove Pa.). The HRP signal was developed with SuperSignal Chemiluminescent Substrate (Pierce). The intensity of the bands was measured by ImageJ (http://rsbweb dot nih dot gov/ij/).

### Cytokine Measurements:

Media from SSc and normal fibroblasts were assayed by ELISA for IL-1β (Thermo Scientific, Rockford Ill.) and IL-18 (MBL, Woburn Mass.) according to the manufacturer's recommendations. The caspase-1 inhibitor Z-YVAD(OMe)-FMK did not interfere with the ELISA assays (data not shown). The sensitivity of the IL-1β ELISA was 2.6 pg/ml and the sensitivity of the IL-18 was 12 pg/ml.

siRNA Targeting of Caspase-1:

80% confluent fibroblasts were cultured in serum free Opti-MEM and transfected with siRNA targeting caspase-1 (Santa Cruz) for 48 h, with an additional transfection of the siRNA duplex after 24 h. Cells were harvested for protein for Western blotting and the media was saved for hydroxyproline and IL-1β and IL-18 measurements and cells were lysed for α-SMA detection.

### Immunofluorescence and Phalloidin Staining:

SSc dermal fibroblasts were cultured in chamber slides to 80% confluency and treated with Z-YVAD(OMe)-FMK for 48 h, air dried then fixed with 4% paraformaldehyde for 20 min. Slides were washed with 1×PBS and blocked with 5% goat serum. Primary antibody to α-SMA (1:100, Clone #1A4, Abcam, Cambridge Mass.) and applied to the cells for 40 min, then washed in PBS. Cy2-conjugated goat-anti-mouse (1:200, Jackson ImmunoResearch, West Grove Pa.) was applied to the cells for 40 min, and then washed. Rhodamine phalloidin (1:100, Cytoskeleton, Denver Colo.) was applied to the cells for 15 min, then the cells were washed with PBS, counterstained with DaPI (Invitrogen), visualized with a Nikon Eclipse 80i epi-fluorescent microscope, and photographed at 400× magnification. All images (red for f-actin, green for α-SMA, and triple for simultaneous detection of f-actin, α-SMA and nuclei) were captured at 6 sec, Gain=2, Gamma=0.5.

Mouse skin was deparaffinized, blocked with 5% donkey serum for 20 min, rinsed briefly in PBS, then rabbit-anti-mouse-IL-1β (1:500, Clone #ABIN103019 Antibodies Online, Atlanta Ga.) was applied for 40 min, then washed in PBS. Donkey-anti-rabbit-Cy3 (1:100, Jackson ImmunoResearch) was applied to the section for 40 min, washed in PBS, mounted with DapI, and viewed with an epi-fluorescent microscope. All images were captured at 7 sec, Gain=1, Gamma=0.5.

### Animal Studies:

All animals were housed in the University Laboratory Animal facility and fed ad libitum. Pathogen free C57Bl/6 mice (n=7) were purchased from Jackson Laboratories. NLRP3−/− (n=8), and ASC−/− (n=4) had a C57BL/6 background. Seven untreated C57BL/6 mice were assessed for skin thickness and lung pathology. Dermal fibrosis was induced by daily subcutaneous administration of 100 μl of a 1 mg/ml of bleomycin (BioMol, Plymouth Meeting Pa.) in the flank for 28 days. At the same time 100 μl sterile PBS was administered by subcutaneous injection in the contralateral flank of the same mouse according to the method of Yamamoto et al. (Yamamoto et al., 1999, J Invest Dermatol 112:456-62). Mice were euthanized and tissue from both injection sites was harvested, along with lung tissue. Tissues were fixed, embedded in paraffin, and sectioned onto positively charged slides, then stained with Masson's Trichrome and photographed. Collagen was measured in the skin from just below the epidermis to the fat layer. Two images were taken at 100× magnification. Six measurements were taken for each image using the Advanced SpotCam software and expressed as skin thickness in microns±SEM and fold change in skin thickness.

### Proliferation Assays:

10,000 cells were seeded into wells in a 96-well plate in 100 μl volume of culture media. Cells were treated with 20 μM Z-YVAD(OMe)-FMK for 48 h, then 10 μl WST-1 reagent (Roche, Indianapolis Ind.) was added and the cells incubated for 4 h at 37° C./5% CO₂. Absorbances were measured at 440 nm and ODs were corrected for background absorbance.

### Array Studies:

The inflammasome RT-PCR arrays were purchased from SABiosciences, Frederick Md. Three SSc dermal fibroblast cell lines (SSc1, SSc3, and SSc4) and three normal dermal fibroblast cell lines (C1, C2, and C3) were assayed according to the manufacturer's protocol. Data was analyzed using http://pcrdataanalysis dot sabiosciences dot com and genes that had a 2-fold change or more in expression as compared to controls were reported.

### Statistics:

Statistical analyses were performed using either a paired 2-tailed student's T-Test where required, or an unpaired 2-tailed student's T-Test and p values less than or equal to 0.05 were considered to be significant.

### Example 1

### Inflammasome Components and Downstream Signaling Molecules are Upregulated in SSc Dermal Fibroblasts

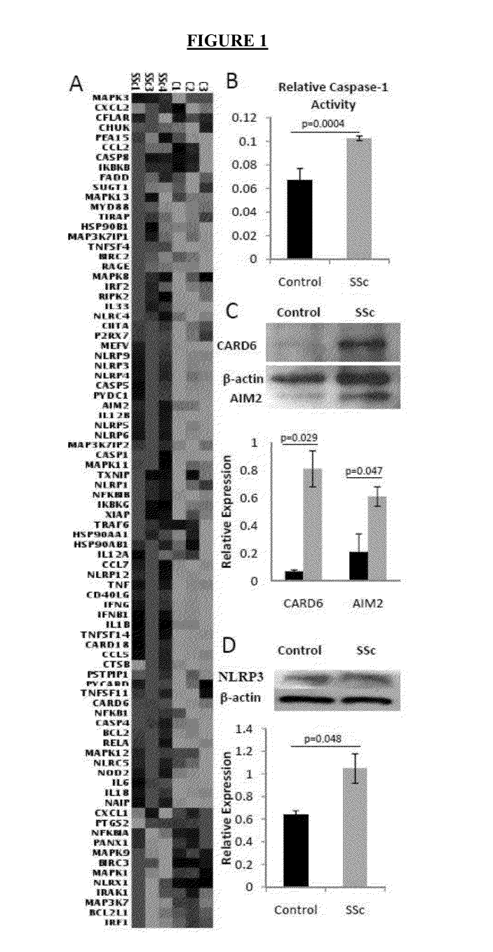

The gene expression of 84 genes involved in inflammasome signaling in three SSc dermal fibroblast lines and three normal dermal fibroblast lines was investigated by RT-PCR. In 8 SSc dermal fibroblasts 40 genes were upregulated more than 2-fold (FIG. 1A and Table 2), whereas only one gene (prostaglandin endoperoxidase 2) was downregulated more than 2-fold, compared to healthy fibroblasts. Activation of AIM2, NLRC4/IPAF, NLRP1, or NLRP3 initiates the formation of distinct inflammasome scaffolds leading to activation of caspase-1 and cleavage of pro-IL-1β and pro-IL-18. AIM2 (p=0.017) and NLRP3 (p=0.028) expression is increased suggesting that at least two inflammasome signaling platforms may be activated in SSc fibroblasts. In addition, increased transcripts of IL-1β (p=0.02) and IL-18 (p=0.0019) were observed, consistent with the increased secretion of IL-1β and IL-18 by SSc fibroblasts. The proinflammatory caspases, caspase-1 (p=0.007), caspase-4 (p=0.0015), and caspase-5 (p=0.043) were elevated in SSc dermal fibroblasts. Five other NLRs were significantly upregulated (NLRP4 (p=0.029), NLRP5 (p=0.017), NLRP6 (p=0.016), NLRP9 (p=0.034), and NLRP12 (p=0.04)), as was NOD2 (p=0.0045) and the assessory molecule RIPK2 (p=0.0089). In addition to increased NLR transcripts, statistically significant increases in the expression of other downstream signaling molecules such as CCL5 (p=0.026), CCL7 (p=0.032), IL-6 (p=0.0047), IL-33 (p=0.025), IFN-β1 (p=0.023), and IFN-γ (p=0.047) were observed. Protein analysis demonstrated that caspase-1 activity was increased almost 2-fold in SSc fibroblasts, 0.103±0.005 vs. 0.068±0.002 in controls, p=0.0004) and CARD6, AIM2 and NLRP3 inflammasome proteins were significantly elevated in SSc; p=0.029, p=0.047, and p=0.048 respectively (FIG. 1B-D).

### Example 2

### IL-1β and IL-18 Secretion by SSc Fibroblasts is Controlled by Caspase-1

In the light of the increased expression of inflammasome transcripts and caspase-1 activity in SSc dermal fibroblasts, the possibility of elevated secretion of IL-1β and IL-18 was investigated. Normal dermal fibroblasts (n=3) secreted 13.6±0.7 pg/ml of IL-1β whereas SSc dermal fibroblasts (n=4) secreted 41.1±5.6 pg/ml (p=0.014, FIG. 2A). When the Z-YVAD(OMe)-FMK inhibitor was added to the SSc dermal fibroblasts, IL-1β was reduced to 12.0±1.6 pg/ml (p=0.012), and was comparable to levels measured in normal dermal fibroblasts (p=0.08; FIG. 2A). Normal lung fibroblasts (n=3) secreted 3.3±0.5 pg/ml of IL-1β whereas SSc lung fibroblasts (n=3) secreted a 6-fold higher concentration, 19.7±0.4 pg/ml (p=0.006; FIG. 2B). In the presence of the caspase-1 inhibitor the secreted IL-1β by SSc lung fibroblasts was reduced to 7.2±0.2 pg/ml (p=0.001; FIG. 3B), within the normal levels of non-fibrotic lung fibroblasts (p=0.2). Caspase-1 siRNA reduced the secretion of IL-1β by SSc dermal fibroblasts from 35.2±3.8 to 9.3±1.2 pg/ml (p=0.02, FIG. 2F).

Likewise, IL-18 secretion in SSc dermal fibroblasts was increased and could be reduced by ZYVAD(OMe)-FMK (FIG. 2C). Normal dermal fibroblasts (n=3) secreted 12.4±2.0 pg/ml of IL-18 whereas SSc fibroblasts (n=4) excreted a 2-fold higher concentration (23.9±1.0 pg/ml; p=0.02). When Z-YVAD(OMe)-FMK was added to the SSc fibroblasts this secretion of IL-18 declined to 15.2±1.5 pg/ml (p=0.008, FIG. 2C), and was not statistically different from normal dermal fibroblasts (p=0.41). Normal lung fibroblasts (n=3) secreted 256.8±23.5 pg/ml of IL-18 whereas SSc lung fibroblasts (n=3) secreted a 3-fold higher concentration 743.9±6.4 pg/ml (p=0.002; FIG. 2D). The addition of Z-YVAD(OMe)-FMK to SSc lung fibroblasts reduced IL-18 secretion to 311.6±18.5 pg/ml (p=0.001, FIG. 2D), but not statistically different from normal lung fibroblasts (p=0.2). Caspase-1 siRNA treatment reduced the secretion of IL-18 by SSc dermal fibroblasts from 78.7±5.6 to 35.7±8.9 pg/ml (p=0.015, FIG. 2G).

### Example 3

### Collagen Expression in SSc Fibroblasts is Regulated by Caspase-1

IL-1β and/or IL-18 can mediate collagen expression via the induction of TGF-β1, therefore the possibility of whether inhibiting caspase-1 with Z-YVAD(OMe)-FMK or siRNA could reduce collagen expression was investigated. Normal dermal fibroblasts (n=3) secreted 9.7±2.19 pg/ml, whereas SSc dermal fibroblasts (n=4) secreted 18.73±2.33 μg/ml hydroxyproline (FIG. 3A; p=0.03). SSc dermal fibroblasts treated with Z-YVAD(OMe)-FMK decreased the secretion of hydroxyproline to 5.19±0.94 μg/ml (p=0.003; FIG. 3A), but not statistically significant when compared with averaged normal dermal fibroblasts (p=0.5).

To further confirm the role of caspase-1 in SSc fibroblast collagen production, the production of COL1A1 and COL3A1 proteins was examined by western blotting. Secreted COL1A1 protein was decreased with the Z-YVAD(OMe)-FMK inhibitor from 11.4±1.3 arbitrary units (AU) to 8.9±1.0 AU (FIG. 3D&E). There was a trend for the reduction in COL1A1 protein (p=0.08). COL3A1 protein expression was found to be more sensitive to the Z-YVAD(OMe)-FMK treatment and decreased from 14.7±3.1 AU (untreated cells) to 8.6±2.5 AU in the treated cells (FIG. 3D&F; p=0.003).

In primary SSc lung fibroblasts (n=3), inhibition of caspase-1 with Z-YVAD(OMe)-FMK also decreased total collagen secreted by the fibroblasts (FIG. 3B). Normal lung fibroblasts (n=3) secreted 52.2±1.0 μg/ml hydroxyproline whereas SSc lung fibroblasts secreted 216.1±18.5 μg/ml hydroxyproline (p=0.01). SSc lung fibroblasts treated with Z-YVAD(OMe)-FMK decreased the secretion of hydroxyproline to 132.0±22.8 μg/ml when compared to untreated SSc lung fibroblasts (p=0.01; FIG. 3B) but not statistically different from normal dermal fibroblasts (p=0.07).

This was further confirmed with western blotting for COL1A1 and COL3A1 proteins. ZYVAD(OMe)-FMK reduced the secretion of COL1A1 and COL3A1 (FIG. 3G-I) in SSc lung fibroblasts; COL3A1 was significantly reduced (12.3±3.8 AU in untreated vs. 5.5±1.2 AU in ZYVAD(OMe)-FMK treated cells; p=0.03, FIG. 3G&I), with a trend for the reduction in COL1A1 (p=0.051, FIG. 3G&H).

To further confirm the role of caspase-1 in collagen secretion by SSc fibroblasts, SSc dermal fibroblasts were treated with siRNA targeting caspase-1. A significant reduction in hydroxyproline secreted by SSc fibroblasts was observed: from 11.9±1.3 μg/ml to 4.7±0.4 μg/ml (p=0.006, FIG. 3C).

### Example 4

### Inhibition of Caspase-1 Decreases α-Smooth Muscle Actin (α-SMA) and Reduces Myofibroblast Morphology

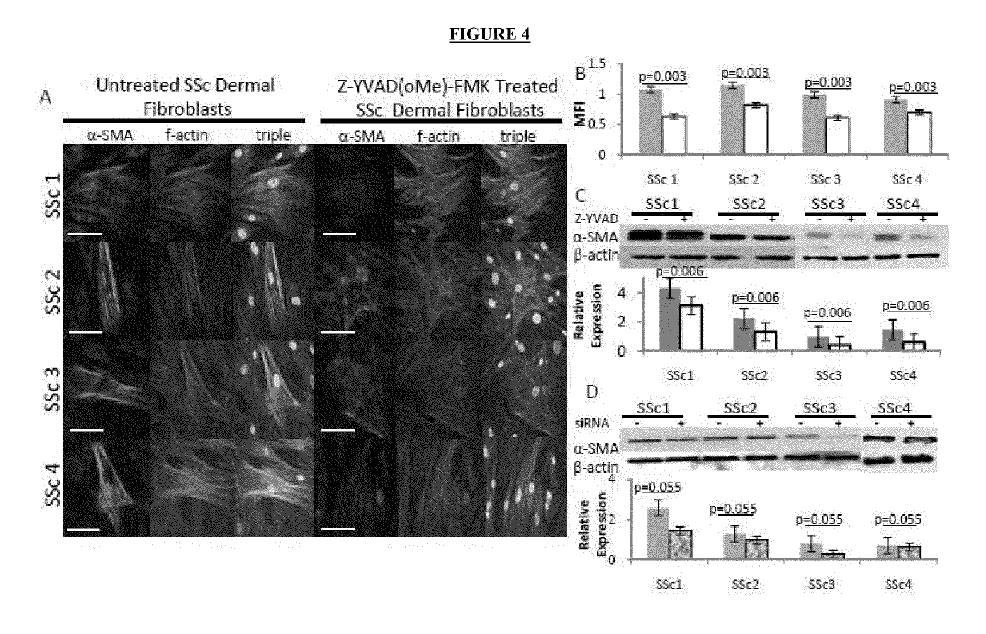

Myofibroblasts secrete excessive levels of collagen in the skin and internal organs in SSc and these cells have a distinct morphology containing discrete α-SMA fibers (Hinz et al., 2003, Mol Biol Cell 14:2508-19). Because there was less collagen, IL-1β, and IL-18 secreted by SSc cells treated with ZYVAD(OMe)-FMK, there was interest in determining if α-SMA was also reduced. The morphology of SSc myofibroblasts was therefore investigated when stained for α-SMA (green) and f-actin (red). Untreated SSc dermal myofibroblasts presented with strongly staining α-SMA and thickened contractile fibers (FIG. 4A). After 48 h treatment with Z-YVAD(OMe)-FMK, the α-SMA expression was decreased and the contractile fibers became less pronounced (FIG. 4A). Mean fluorescent intensity (MFI) of α-SMA in SSc dermal myofibroblasts, after correction for f-actin MFI was 1.03±0.05 and this declined to 0.68±0.04 (p=0.003; FIG. 4B) with the caspase-1 inhibitor. F-actin staining was unaffected by Z-YVAD(OMe)-FMK treatment and the cytoskeleton of the myofibroblasts and fibroblasts was as pronounced (37.8±3.09 MFI in untreated vs. 39.6±2.9 MFI in treated cells, p=0.11). By Western, α-SMA protein was decreased in Z-YVAD(OMe)-FMK treated SSc fibroblasts (1.07±0.15 in untreated vs. 0.54±0.14 in treated cells, p=0.006, FIG. 4C), with a statistical trend for decreased α-SMA with caspase-1 siRNA (p=0.055, FIG. 4D).

### Example 5

### The Inflammasome Participates in Bleomycin Induced Dermal Fibrosis

The role of the inflammasome proteins in dermal fibrosis in vivo was investigated by utilizing the murine bleomycin-induced model of fibrosis. Using this model, both the NLRP3 and ASC proteins were found to be absolutely necessary for the development of dermal fibrosis. As expected C57Bl/6 mice had increased collagen deposition with marked skin thickening induced by bleomycin. In the PBS injected skin, average skin thickness was 320.7±40.6 microns whereas in the bleomycin injected skin it was 504.4±66.7 microns (1.54±0.11 fold increase in thickness; p=0.043). In the skin of the untreated mice, average skin thickness was found to be 279.3±20.3 and not to be statistically different from the PBS injected skin (p=0.37). The bleomycin induction of skin thickness was abolished in the skin of NLRP3−/− (1.10 fold; p=0.002) and ASC−/− (1.06 fold; p=0.01; FIG. 5) when compared to C57Bl/6 bleomycin injected skin. The maintenance of pulmonary architecture and structure was unaffected in the NLRP3−/− and ASC−/− mice, whereas wild-type C57Bl/6 mice developed extensive pulmonary fibrosis characterized by alveolar occlusion and collagen deposition leading to a pronounced loss of tissue structure and function (FIG. 5). In further analysis of fibroblasts established from the skin of the mice, they were stimulated with bleomycin and caspase-1 activation was blocked with Z-YVAD(OMe)-FMK, and hydroxyproline was measured in the media (FIG. 5C). C57BL/6 fibroblasts secreted 10.6±3.8 μg/ml hydroxyproline and when stimulated with 1 μM bleomycin for 48 h secreted 42.8±2.9 μg/ml hydroxyproline (p=0.036) and this could be inhibited by Z-YVAD(OMe)-FMK (5.9±0.7 μg/ml hydroxyproline, p=0.008 when compared to bleomycin stimulated cells). However in the ASC−/− and NLRP3−/− fibroblasts, hydroxyproline secretion was not induced (p>0.43; FIG. 5C). These results suggest that the inflammasome and caspase-1 activation is required for bleomycin induced in vivo skin fibrosis and in vitro collagen secretion.

### Example 6

The inflammasome is involved in the detection of intracellular microbial products and host-derived danger signals, and are key intracellular sensors in the innate immune response. In addition to their primary role in host defense, they are also involved in regulating cell death, as well as IL-1β and IL-18 processing, and are crucial signaling molecules in a wide variety of inflammatory diseases. As demonstrated herein, inflammasome proteins and proteins involved in inflammasome signaling are increased in SSc fibroblasts. Further, experiments performed herein indicated increased expression of NOD2, AIM2, and NLRP3, by inflammasomes. AIM2 is an intracellular sensor of cytosolic double-stranded DNA, bacterial DNA and DNA viruses and once activated leads to the cleavage of pro-caspase-1. Further, increased expression of several other NLRP genes (NLRP4, NLRP5, NLRP6, NLRP9, and NLRP12) in SSc fibroblasts was also evidenced. NLRP12 is known to be important in cutaneous inflammation but thought not to be involved in IL-1β processing. However, there was no increase in NLRP1 expression where genetic associations have been implicated in SSc. Without wishing to be limited by theory, this disconnect may be due to the location of the gene association in NLRP1 as there may be gain of function for this inflammasome rather than an increase in its expression.

NOD2 engages with RIPK2 (both of which were found to have increased expression in SSc dermal fibroblasts; Table 2), and RIPK2 also senses intracellular muramyl dipeptide from Gram-positive bacteria to induce the upregulation of proinflammatory and antimicrobial responses independent of TLR. Not only is the RIPK2 signaling pathway required for NOD2 to sense intercellular pathogens, it is also required for the efficient recruitment of antigen-specific T and B cells and is necessary for the polarization of TH1 and TH2, and TH17 pathways by regulating the expression of IL-23, IL-12, interferon-γ and IL-17; cytokines known to be increased in SSc sera. Furthermore, this observation suggests that the specificity of the NLR receptors that are activated may be more important in mediating the downstream signaling events that promote fibrosis. The identification of at least three defined inflammasome signaling platforms involved in primary SSc dermal fibroblasts suggests a role for the inflammasome in fibrosis and SSc pathogenesis in at least those patients with diffuse disease. Studies on other subsets of disease e.g. limited SSc or morphea could identify potential signaling differences mediated by altered patterns of NLR expression.

With a link between IL-1β and the inflammasome well-established in idiopathic pulmonary fibrosis, the contribution of the inflammasome within dermal and lung fibroblasts in diffuse SSc disease was investigated. SSc dermal and lung fibroblasts had activated caspase-1 as the addition of Z-YVAD(OMe)-FMK abrogated collagen, IL-1β, and IL-18 protein secretion. Inhibition of caspase-1 (both chemically and siRNA) significantly decreased hydroxyproline secretion. Additionally, Z-YVAD(OMe)-FMK and siRNA for caspase-1 mediated a decrease in α-SMA expression in SSc myofibroblasts resulting in fewer and thinner stress fibers within the cell. Taken together, the experimental results suggested that activation of caspase-1 and the down-stream signaling cascade and cytokine production mediates fibrosis in the dermis and lung in the diffuse form of SSc.

Using NLRP3 and ASC deficient mice, the role of the inflammasome and caspase-1 activation in dermal fibrosis was further investigated in vivo. Bleomycin induced pulmonary fibrosis had been previously linked to IL-1 cytokine family (Gasse et al., 2007, J Clin Invest 117:3786-99); however, an association between the NLRP3 inflammasome and dermal fibrosis has not been previously described. In vivo, dermal administration of bleomycin was performed in NLRP3−/− and ASC−/− mice and compared to bleomycin induced skin thickening in the wild-type C57BL/6 mice. In response to bleomycin, C57BL/6 wild-type mice developed marked dermal thickening due to heavy collagen deposition. Analysis of the lung tissue from C57BL/6 revealed the systemic effect of bleomycin as the lung architecture was severely compromised with increased collagen deposition and loss of alveoli structure. In contrast, dermal fibrosis was not induced in NLRP3−/− or ASC−/− mice and lung architecture was normal. This result strongly suggests that the NLRP3 inflammasome and the down-stream signaling cascade mediate the fibrotic processes in bleomycin induced skin and lung fibrosis in mice. These studies were confirmed in fibroblast lines established from C57BL/6, ASC−/− and NLRP3−/− mice. ASC and NLRP3 deficiency inhibited bleomycin induced induction of hydroxyproline secretion by these cells. Caspase-1 inhibition in bleomycin treated wild-type fibroblasts also reduced hydroxyproline production.

Taken together the above indicate that both in vivo and in vitro bleomycin induced fibrosis is inflammasome mediated. In SSc, fibrosis directly correlates with morbidity and mortality, and in this study inflammasome signaling mediated by active caspase-1 in fibroblasts from SSc patients with the diffuse form of the disease was investigated. The activation of the inflammasome mediating pro-caspase-1 cleavage resulting in IL-1β and IL-18 secretion and the down-stream activation of the innate immune response in SSc suggests that IL-1β and/or IL-18 may be driving fibrosis. The myofibroblast is believed to be the pathologic cell that promotes fibrosis in the skin and visceral organs as it secretes excessive amounts of collagen and is characterized by stress fibers. Inhibiting caspase-1 ameliorated the myofibroblast phenotype suggesting that there may be autocrine signaling possibly via IL-1β or IL-18 that maintains the activated phenotype of this cell.

The etiology of SSc remains unknown and therefore the mechanism whereby fibrosis is established in this disease still remains elusive, however exposure to infectious disease (Radic et al., 2010 Neth J Med 68:348-53) and chemicals (Haustein et al., 1994, Clin Dermatol 12:467-73) have been implicated and may be mediating the increased activity in the inflammasome in fibroblasts. Because the inflammasome platforms are intracellular sensors for cellular injury, are integral to the host defense mechanisms to eradicate infection, and because inflammasome activation of capsase-1 and downstream signaling molecules mediated by this activation can induce fibrosis in the diffuse SSc dermal and lung fibroblasts SSc etiology may be mediated by a latent and persistent infection or exposure to an environmental factor that triggers the inflammasome pathway. The possible role of an etiological factor may further highlight the relevance between the inflammasome and the autoimmune disease SSc. The studies presented herein also suggest that inflammasome and downstream mediators may be useful targets for therapeutic intervention.

TABLE 2
Expression of Inflammasome Gene Transcripts in SSc Dermal Fibroblasts: Three SSc dermal fibroblast lines (SSc1, SSc3,
SSc4) were compared to three normal dermal fibroblast lines (C1, C2, C3) and gene expression is listed in descending order.

| Gene | | Fold | P | Accession |
|---|---|---|---|---|
| Symbol | Gene Name | Change | value | # |
| CCL2 | Chemokine (C-C motif) ligand 2 (MCP-1) | 13.82 | 0.963 | NM_002982 |
| CARD6 | Caspase recruitment domain family, member 6 | 13.72 | 0.00002 | NM_032587 |
| AIM2 | Absent in melanoma 2 | 11.79 | 0.017 | NM_004833 |
| IL6 | Interleukin 6 (interferon β2) | 8.69 | 0.0047 | NM_000600 |
| NLRP5 | NLR family, pyrin domain containing 5 | 8.56 | 0.017 | NM_153447 |
| PYDC1 | PYD (pyrin domain) containing 1 | 8.32 | 0.042 | NM_152901 |
| CCL5 | Chemokine (C-C motif) ligand 5 (RANTES) | 8.28 | 0.026 | NM_002985 |
| IFNG | Interferon γ | 8.23 | 0.047 | NM_000619 |
| CASP5 | Caspase-5, apoptosis-related cysteine peptidase | 7.89 | 0.043 | NM_004347 |
| CASP8 | Caspase-8, apoptosis-related cysteine peptidase | 7.83 | 0.339 | NM_001228 |
| TNFSF4 | Tumor necrosis factor (ligand) superfamily, member 4 | 7.57 | 0.114 | NM_330026 |
| BCL2 | B-cell CLL/lymphoma 2 | 7.55 | 0.0005 | NM_000633 |
| NLRP4 | NLR family, pyrin domain containing 4 | 7.48 | 0.029 | NM_134444 |
| IL1B | Interleukin 1β | 7.14 | 0.02 | NM_000576 |
| NLRP3 | NLR family, pyrin domain containing 3 | 7.02 | 0.028 | NM_183395 |
| MEFV | Mediterranean fever, pyrin | 6.91 | 0.035 | NM_000243 |
| IFNB1 | Interferon β1, fibroblast | 6.78 | 0.023 | NM_002176 |
| NLRP12 | NLR family, pyrin domain containing 12 | 6.60 | 0.040 | NM_033297 |
| CARD18 | Caspase recruitment domain family, member 18 | 6.56 | 0.032 | NM_021571 |
| NLRP6 | NLR family, pyrin domain containing 6 | 6.28 | 0.016 | NM_138329 |
| NOD2 | Nucleotide-binding oligomerization domain containing 2 | 6.08 | 0.0045 | NM_022162 |
| TNF | Tumor necrosis factor (TNF superfamily, member 2) | 6.03 | 0.035 | NM_000594 |
| CASP1 | Caspase-1, apoptosis-related cysteine peptidase (interleukin 1β convertase) | 5.71 | 0.007 | NM_033292 |
| CD40LG | CD40 ligand | 5.48 | 0.064 | NM_000074 |
| IL12B | Interleukin 12B (natural killer stimulatory factor 2) | 5.20 | 0.014 | NM_002187 |
| IL18 | Interleukin 18 (Interferon γ-inducing factor) | 5.02 | 0.0019 | NM_001562 |
| TNFSF14 | Tumor necrosis factor (ligand) superfamily, member 14 | 4.38 | 0.017 | NM_003807 |
| CCL7 | Chemokine (C-C motif) ligand 7 (MCP3) | 4.19 | 0.032 | NM_006273 |
| IL33 | Interleukin 33 | 4.13 | 0.025 | NM_033439 |
| HSP90AB1 | Heat shock protein 90 kDA alpha (cytosolic), class B member 1 | 3.37 | 0.037 | NM_007355 |
| NAIP | NLR family, apoptosis inhibitory protein | 3.33 | 0.011 | NM_004536 |
| RAGE | Receptor for advanced glycation end products | 2.82 | 0.250 | NM_014226 |
| TNFSF11 | Tumor necrosis factor (ligand) superfamily, member 11 | 2.71 | 0.016 | NM_003701 |
| CASP4 | Caspase-4, apoptosis-related cysteine peptidase | 2.70 | 0.0015 | NM_001225 |
| CIITA | Class II, major histocompatibility complex, transactivator | 2.67 | 0.047 | NM_000246 |
| NLRP9 | NLR family, pyrin domain containing 9 | 2.63 | 0.034 | NM_176820 |
| NFKBIB | Nuclear factor of kappa light polypeptide gene enhancer in B-cells inhibitor β | 2.46 | 0.028 | NM_002503 |
| RIPK2 | Receptor-interacting serine-theorine kinase-2 | 2.32 | 0.0089 | NM_003821 |
| XIAP | X-linked inhibitor of apoptosis | 2.25 | 0.05 | NM_001167 |
| NFKB1 | Nuclear factor of kappa light polypeptide gene enhancer in B-cells 1 | 2.08 | 0.0007 | NM_003998 |
| MAPK12 | Mitogen-activated protein kinase 12 | 2.00 | 0.0042 | NM_002969 |
| MAPK11 | Mitogen-activated protein kinase 11 | 1.97 | 0.013 | NM_002571 |
| RELA | ν-rel reticuloendotheliosis viral oncogene homolog A | 1.89 | 0.002 | NM_021975 |
| P2RX7 | Purinergic receptor P2X, ligand-gated ion channel, 7 | 1.87 | 0.07 | NM_002562 |
| MAPK8 | Mitogen-activated protein kinase 8 | 1.80 | 0.044 | NM_002750 |
| NLRC4 | NLR family, CARD domain containing 4 | 1.77 | 0.013 | NM_021209 |
| TRAF6 | TNF receptor-associated factor 6 | 1.73 | 0.67 | NM_004620 |
| CHUK | Conserved helix-loop-helix ubiquitous kinase | 1.67 | 0.37 | NM_001278 |
| MAP3K7IP2 | TGF-beta activated kinase 1/MAP3K7 binding protein 2 | 1.65 | 0.26 | NM_015093 |
| IKBKG | Inhibitor of kappa light polypeptide gene enhancer in B-cells, kinase gamma | 1.63 | 0.018 | NM_003639 |
| NLRC5 | NLR family, CARD domain containing 5 | 1.55 | 0.004 | NM_032206 |
| PSTPIP1 | Proline-serine-threonine phosphatase interacting protein 1 | 1.55 | 0.075 | NM_003978 |
| IRF2 | Interferon regulatory factor 2 | 1.47 | 0.077 | NM_002199 |
| CTSB | Cathepsin B | 1.44 | 0.201 | NM_001908 |
| FADD | Fas (TNFRSF6)-associated via death domain | 1.42 | 0.233 | NM_003824 |
| TXNIP | Thioredoxin interacting protein | 1.42 | 0.091 | NM_006472 |
| HSP90B1 | Heat shock protein 90 kDa beta (Grp94), member 1 | 1.41 | 0.149 | NM_003299 |
| MAP3K7IP1 | TGF-beta activated kinase 1/MAP3K7 binding protein 1 | 1.4 | 0.138 | NM_006116 |
| PYCARD | PYD and CARD domain containing protein | 1.33 | 0.020 | NM_013258 |
| HSP90AA1 | Heat shock protein 90 kDa alpha (cytosolic), class A member 1 | 1.31 | 0.009 | NM_001017693 |
| TIRAP | Toll-interleukin 1 receptor (TIR) domain containing adaptor protein | 1.26 | 0.249 | NM_001039661 |
| NLRP1 | NLR family, pyrin domain containing 1 | 1.25 | 0.116 | NM_033004 |
| BIRC2 | Baculoviral IAP repeat-containing 2 | 1.24 | 0.336 | NM_001166 |
| IKBKB | Inhibitor of kappa light-polypeptide gene enhancer in B-cells, kinase beta | 1.24 | 0.316 | NM_001556 |
| MAPK13 | Mitogen-activated protein kinase 13 | 1.23 | 0.327 | NM_002754 |
| MYD88 | Myeloid differentiation primary response gene 88 | 1.23 | 0.304 | NM_002468 |
| IL12A | Interleukin 12A | 1.22 | 0.226 | NM_000882 |
| CXCL2 | Chemokine (C-X-C motif) ligand 2 | 1.16 | 0.439 | NM_002089 |
| SUGT1 | SGT1, suppressor of G2 allele of SKP1 | 1.12 | 0.544 | NM_006704 |
| PEA15 | Phosphoprotein enriched in astrocytes 15 | 1.11 | 0.692 | NM_003768 |
| MAPK3 | Mitogen-activated protein kinase 3 | 1.09 | 0.686 | NM_002746 |
| IRAK1 | Interleukin-1 receptor-associated kinase 1 | 1.07 | 0.685 | NM_001569 |
| IRF1 | Interferon regulatory factor 1 | 1.03 | 0.756 | NM_002198 |
| MAP3K7 | Mitogen-activated protein kinase kinase kinase 7 | 1.03 | 0.768 | NM_003188 |
| BCL2L1 | BCL2-like 1 | 1.00 | 0.704 | NM_138578 |
| MAPK1 | Mitogen-activated protein kinase 1 | −1.05 | 0.929 | NM_002745 |
| CFLAR | CASP8 and FADD-like apoptosis regulator | −1.06 | 0.974 | NM_003879 |
| NLRX1 | NLR family member X1 | −1.11 | 0.765 | NM_024618 |
| MAPK9 | Mitogen-activated protein kinase 9 | −1.16 | 0.436 | NM_002752 |
| BIRC3 | Baculoviral IAP repeat-containing 3 | −1.34 | 0.673 | NM_001165 |
| CXCL1 | Chemokine (C-X-C motif) ligand 1 | −1.48 | 0.037 | NM_001511 |
| NFKBIA | Nuclear factor of kappa light polypeptide gene enhancer in B-cells inhibitor α | −1.50 | 0.160 | NM_020529 |
| PANX1 | Pannexin 1 | −1.65 | 0.116 | NM_015368 |
| PTGS2 | Prostaglandin-endoperoxide synthase 2 | −4.87 | 0.0022 | NM_000963 |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

SEQ ID NOs:

IL-1β
SEQ ID NO: 1
APVRSLNCTL RDSQQKSLVM SGPYELKALH LQGQDMEQQV
VFSMSFVQGE ESNDKIPVAL GLKEKNLYLS CVLKDDKPTL
QLESVDPKNY PKKKMEKRFV FNKIEINNKL EFESAQFPNW
YISTSQAENM PVFLGGTKGG QDITDFTMQF VSS

What is claimed:

1. A method of treating or preventing fibrosis in a subject afflicted with scleroderma, the method comprising administering to the subject a therapeutically effective amount of a pharmaceutical composition comprising an agent that inhibits formation of at least one inflammasome signaling product in the subject, whereby the fibrosis in the subject is treated or prevented.

2. The method of claim 1, wherein the agent that inhibits formation of at least one inflammasome signaling product is selected from the group consisting of glyburide, parthenolide, BAY 11-7082, colchicine, a caspase-1 inhibitor, an IL-1β-depleting agent, and any combinations thereof.

3. The method of claim 2, wherein the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, a ribozyme, an antisense, an aptamer, a peptidomimetic, a small molecule, and any combinations thereof.

4. The method of claim 3, wherein the IL-1β antibody comprises an antibody selected from the group consisting of a polyclonal antibody, monoclonal antibody, humanized antibody, synthetic antibody, heavy chain antibody, human antibody, biologically active fragment of an antibody, and any combinations thereof.

5. The method of claim 1, wherein the caspase-1 inhibitor is selected from the group consisting of a caspase-1 antibody, siRNA, ribozyme, antisense, aptamer, peptidomimetic, small molecule, and a combination thereof.

6. The method of claim 5, wherein the caspase-1 antibody comprises an antibody selected from the group consisting of a polyclonal antibody, monoclonal antibody, humanized antibody, synthetic antibody, heavy chain antibody, human antibody, biologically active fragment of an antibody, and any combinations thereof.

7. The method of claim 1, comprising further administering to the subject an additional anti-scleroderma composition.

8. The method of claim 7, wherein the anti-scleroderma composition comprises a vasodilator, an endothelin receptor antagonist, an angiotensin converting enzyme inhibitor, an immunosuppressive agent, halofuginone, CAT-192, cyclophosphamide and rabbit antithymocyte globulins, dasatinib, fludarabine, imatinib mesylate (Gleevec), nilotnib (Tasigna) or rituximab.

9. The method of claim 8, wherein the agent and the anti-scleroderma composition are co-administered to the subject.

10. The method of claim 9, wherein the agent and the anti-scleroderma composition are co-formulated and co-administered to the subject.

11. The method of claim 1, wherein the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combination thereof.

12. The method of claim 1, wherein the subject is a mammal.

13. The method of claim 12, wherein the mammal is a human.

14. A kit comprising an agent that inhibits formation of at least one inflammasome signaling product in a subject, an applicator, and an instructional material for use thereof,
wherein the agent that inhibits formation of at least one inflammasome signaling product is selected from the group consisting of glyburide, parthenolide, BAY 11-7082, colchicine, a caspase-1 inhibitor, an IL-1β-depleting agent, and any combinations thereof;
wherein the instructional material comprises instructions for preventing or treating fibrosis in a subject afflicted with scleroderma,
wherein the instructional material recites that the agent is to be administered to the subject in an amount sufficient to inhibit formation of at least one inflammasome signaling product in the subject, whereby the fibrosis in the subject is treated or prevented.

15. The kit of claim 14, wherein the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, ribozyme, an antisense, an aptamer, a peptidomimetic, a small molecule, and any combination thereof.

16. The kit of claim 14, wherein the caspase-1 inhibitor is selected from the group consisting of a caspase-1 antibody, siRNA, ribozyme, antisense, aptamer, peptidomimetic, small molecule, and a combination thereof.

17. The kit of claim 14, wherein the subject is a mammal.

18. The kit of claim 17, wherein the mammal is human.


SEQUENCE LISTING (1 sequence)

SEQ ID NO: 1 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS